(19) United States
(12) Patent Application Publication
(10) Pub. No.: US 2026/0356865 A1
(43) Pub. Date: Nov. 12, 2026

(54) IMPROVED SYNTHESIS OF PSILOCYBIN DERIVATIVES

(71) Applicants: CARBOGEN AMCIS AG, Bubendorf (CH); AAMANYA AG, Vitznau (CH)

(72) Inventors: Christian HAMMES, Bubendorf (CH); Stefan STÖCKLI, Bubendorf (CH)

(21) Appl. No.: 19/107,442

(22) PCT Filed: Sep. 21, 2023
(86) PCT No.: PCT/EP2023/076160

§ 371 Date: Feb. 27, 2025

(30) Foreign Application Priority Data
Sep. 21, 2022 (EP) 22196997.5

(51) Int. Cl. C07F 9/572; C07D 209/08; C07D 209/16; C07D 209/18

(52) U.S. Cl. CPC C07F 9/5728; C07D 209/08; C07D 209/16; C07D 209/18

(57) ABSTRACT

The present invention relates to an improved synthesis of psilocybin and alkyl-derivatives of psilocybin such as ethocybin (4-phosphoryloxy-N,N-diethyltryptamine, also known as phosphoryloxy-DET, PO-DET or CEY-39) and furthermore, relates to intermediates useful in the synthesis of these compounds.

### BACKGROUND OF THE INVENTION

The present invention relates to an improved synthesis of psilocybin and alkyl-derivatives of psilocybin (IUPAC name: 4-phosphoryloxy-N,N-dimethyltryptamine 3-[2-(dimethylamino)ethyl]-1H-indole-4yl-dihydrogenphosphate) such as e.g. ethocybin (IUPAC name: phosphoric acid mono-[3-(2-dietylamino-ethyl)-1H-indol-4yl] ester) and furthermore, relates to intermediates useful in the synthesis of these compounds.

Psilocybin and its derivatives such as its dephosphorylated active metabolite psilocin have recently come into renewed interest because of their potential usefulness in the treatment of various psychological diseases (Dinis-Olivera R. J. Drug Metab. Rev. 2017, 49 (1), 84-91). For example, said compounds have been demonstrated to be useful for the treatment of depression.

Psilocybin has first been isolated from the mushroom P. mexicana in the year 1958 in the laboratory of Albert Hoffmann (Hofmann, A., Frey, A., Ott, H., Petrzilika, F., Troxler, F.; Experientia 14, 1958, 397-399). Shirota et al published more recently a concise large-scale synthesis of psilocin and psilocybin without a chromatographic purification step (Shirota O., Hakamata, W. and Goda Y.; J. Nat. Prod. 2003, 66 (6), 885-887).

The synthesis of alkyl derivatives of psilocybin such as ethocybin (4-phosphoryloxy-N, N-diethyltryptamine), also known as phosphoryloxy-DET, PO-DET or CEY-39; has been disclosed in U.S. Pat. No. 3,075,992.

Macor, Post and Ryan disclose a simple synthesis of 5-amino-3-(2-dimethylaminoethyl) indole (https://doi.org/10.1080/00397919308020402).

However, when the synthesis procedures as described in the prior art were used to prepare these compounds in a larger scale, low overall yields are obtained. Furthermore, the scale-up is significantly hampered by the required chromatographic purification steps, which are more difficult to be implemented at said scale. There has thus been a need for alternative synthesis procedures which allow higher overall yields especially when performing the synthesis at a multigram or kilogram scale, in particular compliant with the good manufacturing practice requirements.

Document WO 2022/016289 discloses a method for preparing psilocybin starting from psilocin and (tert-BuO)₂POCl. The process disclosed in WO 2022/016289 requires in certain embodiments the use of CCl₄, which makes a scale-up of the reaction and its industrial application less favourable.

Troxler F. et al. (Helvetica Chimica Acta, vol. 42, no. 6, pages 207302193) discloses phosphorylation of hydroxyindol derivatives using dibenzylphosphoryl chloride.

The object of the present invention is therefore the provision of a novel process for the preparation of alkyl derivatives of psilocybin such as ethocybin with an improved overall yield especially when performing the synthesis at a multigram or kilogram scale while avoiding chromatographic purifications.

The present inventors have found that surprisingly the said object can be achieved by synthesis methods as outlined in the present patent application and as claimed in the appended set of claims.

The present invention therefore provides a method for preparing a compound of formula (I):

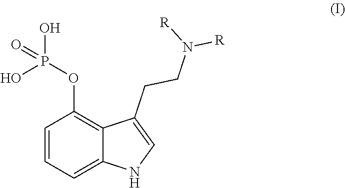

wherein each R is independently C₁₋₆ alkyl (preferably methyl or ethyl),
wherein the method comprises the step of reacting the compound of formula (Id):

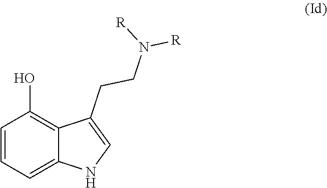

with (EtO)₂POCl (DECP). It is preferred that the product of the reaction of the compound of formula (Id) with DECP is further reacted with trimethylsilylbromide (TMSBr) resulting in the compound of formula (I).

In a preferred method in accordance with the invention R is methyl.

In one embodiment of the invention the w/w ratio of the compound of formula (Id) and (DECP) is between 0.8 and 1.2, preferably between 1.0 and 1.2, more preferably between 1.10 and 1.15, even more preferably about 1.12, even more preferably 1.12.

In another embodiment of the invention DECP is added dropwise over the time of between 10 and 20 minutes.

In a further embodiment of the invention the reaction of the compound of formula (Id) with DECP is performed in acetonitrile.

In a further embodiment of the invention the reaction of the compound of formula (Id) with DECP is performed at a temperature of between 50 °C and 70 °C, preferably at a temperature of between 55 °C and 65 °C, more preferably at a temperature of between 57 °C and 63 °C, even more preferably at a temperature of about 60 °C, even more preferably at a temperature of 60 °C.

In a further embodiment of the invention the reaction of the compound of formula (Id) with DECP is performed in the presence of a base, preferably wherein the base is N-ethyl-diisopropylamine.

In a further embodiment of the invention the reaction of the compound of formula (Id) with DECP is performed for a time of at least 150 minutes, until at least 99% of the compound of formula (Id) has been reacted.

In a further embodiment of the invention the reaction of the compound of formula (Id) with DECP, the solvent is changed to toluene and aqueous NaOH solution is added thereto, followed by the phase separation. Preferably NaOH is added at the concentration of 0.5 M to 1.0 M, preferably at the concentration of about 0.75 M, more preferably at the concentration of 0.75 M.

In a further embodiment of the invention the method upon completion of the reaction of the compound of formula (Id) with DECP, the solvent is changed to cyclopentylmethylether (CPME) and HCl in CPME is added to the reaction mixture, leading to precipitation of a crude product. In a preferred embodiment HCl in CPME is about 3M solution, and/or wherein said solution is added dropwise over a time of between 25 and 35 minutes, and/or wherein said solution is added at a temperature of between 15 °C and 20 °C, preferably at a temperature of between 17 °C and 23 °C, more preferably at a temperature of about 20 °C, even more preferably at a temperature of 20 °C.

In another aspect of the present invention the reaction of the compound of formula (Id) with DECP results in a crude product of formula (Ie):

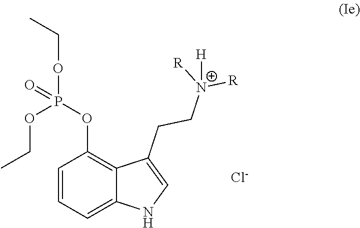

In a preferred embodiment of the invention the crude product precipitated from CPME upon addition of HCl comprises a compound of formula (Ie). In formula (Ie), each R is as defined for formula (I).

It is to be understood that in the embodiments of the present invention wherein the compound of formula (Id) is reacted with DECP, it is recognizable to the skilled person that DECP can be replaced with a compound of formula (R′O)₂POCl, wherein each R′ is independently selected from C₁₋₆ alkyl, —CH₂-aryl or —CH₂-heteroaryl, preferably wherein each R′ is independently selected from C₁₋₆ alkyl and —CH₂-aryl, more preferably wherein each R′ is independently C₁₋₆ alkyl. Accordingly, within the scope of the present invention, in the first step of the method of the present invention, said compound of formula (Id) can be reacted with a compound of formula (R′O)₂POCl, wherein each R′ is independently selected from C₁₋₆ alkyl, —CH₂-aryl or —CH₂-heteroaryl, preferably selected from C₁₋₆ alkyl and —CH₂-aryl. Particularly suitable C₁₋₆ alkyl groups are ethyl and tert-butyl. Particularly suitable —CH₂-aryl is benzyl.

In a further embodiment of the invention the product of the reaction of the compound of formula (Id) with DECP is further reacted with trimethylsilylbromide (TMSBr) resulting in the compound of formula (I), whereby preferably the w/w ratio of TMSBr to the compound of formula (Id) is between 1.5 and 2.5, preferably between 1.8 to 2.4, more preferably between 2.0 and 2.3, even more preferably is about 2.1, even more preferably is 2.07.

In a further embodiment of the invention the product of the reaction of the compound of formula (Id) with diethyl chlorophosphate (DECP) is reacted with TMSBr in acetonitrile, whereby preferably TMSBr is added at a temperature of between 30 °C to 50 °C, preferably at a temperature of between 35 °C and 45 °C, more preferably at a temperature of between 37 °C and 43 °C, even more preferably at a temperature of about 40 °C, even more preferably at a temperature of 40 °C.

In a further embodiment of the invention the product of the reaction of the compound of formula (Id) with DECP is reacted with TMSBr at a temperature of between 50 °C and 70 °C, preferably at a temperature of between 55 °C and 65 °C, more preferably at a temperature of between 57 °C and 63 °C, even more preferably at a temperature of about 60 °C, even more preferably at a temperature of 60 °C.

In a further preferred embodiment of the invention wherein the reaction with TMSBr is conducted for at least 150 minutes, until the product of the reaction of the compound of formula (Id) with DECP is consumed in at least 99%.

In a further preferred embodiment of the invention upon reaction with TMSBr, the solvent is changed to methanol, whereby preferably the methanol is removed by distillation and the remaining solid residue is dissolved in methanol, and the so obtained solution treated with active carbon and filtered.

In a further embodiment of the invention wherein the obtained product of the reaction with TMSBr is subjected to solvent change to water, wherein the pH of the solution is set to a value in the range between pH=3.8 and pH=4.2, preferably through addition of 1 M NaOH.

In a preferred embodiment of the invention the w/w ratio of 1M NaOH solution to the original amount of the compound of formula (Id) added is about 4.0, preferably is 4.0.

In a further embodiment of the invention wherein the final product of formula (I) precipitates from aqueous solution at pH of between 3.8 and 4.2 and/or at a temperature of less than 20 °C, preferably at a temperature about 15 °C, more preferably at a temperature of 15 °C.

In a further embodiment of the invention the final product of formula (I) is treated with water/acetone under reflux.

In a further embodiment of the invention the method further comprises the step of reducing a compound of formula (Ic):

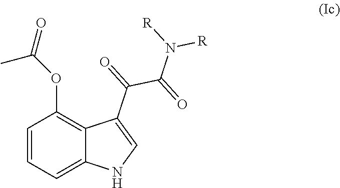

resulting in the compound of formula (Id), whereby preferably in the step of reducing the compound of formula (Ic) resulting in the compound of formula (Id), LAH is used as a reducing agent, whereby in a preferred embodiment the step of reducing the compound of formula (Ic) resulting in the compound of formula (Id) is performed in toluene, in dioxane or in CPME, preferably in toluene, more preferably said step is performed under reflux. In formula (Ic), each R is as defined for formula (I).

In a further preferred embodiment of the invention in the step of reducing the compound of formula (Ic) resulting in the compound of formula (Id), NaBH₄ is used as a reducing agent.

In a further preferred embodiment of the invention the method further comprises the step of reacting a compound of formula (Ia):

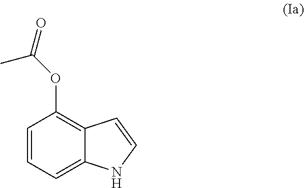

with oxalyl chloride (COCl)₂, followed by the reaction of the resulting product with dialkylamine (R₂NH), e.g. with diethylamine. In R₂NH, each R is as defined for formula (I).

In a further preferred embodiment of the invention the reaction of the compound of formula (Ia) with (COCl)₂ is performed in CPME at a temperature of between 0 °C and 10 °C.

In a further preferred embodiment of the invention the said reaction of the compound of formula (Ia) with (COCl)₂ results in a compound of formula (Ib):

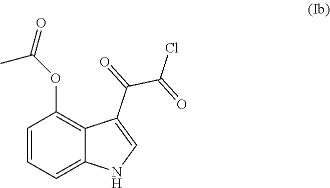 [SMILES: CC(=O)Oc1cccc2[nH]cc(C(=O)C(=O)Cl)c12]

wherein said compound of formula (Ib) is reacted with R₂NH to result in the compound of formula (Ic). In R₂NH, each R is as defined for formula (I).

The present invention will be more fully understood by reference to the following Examples. These Examples should however not be construed as limiting the scope of the invention.

The experimental methods described in the Example section below are further illustrated by the appended figures, which show the following apparatus setups.

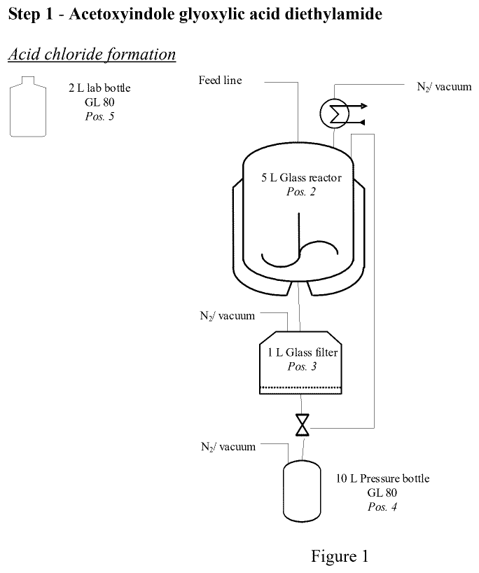

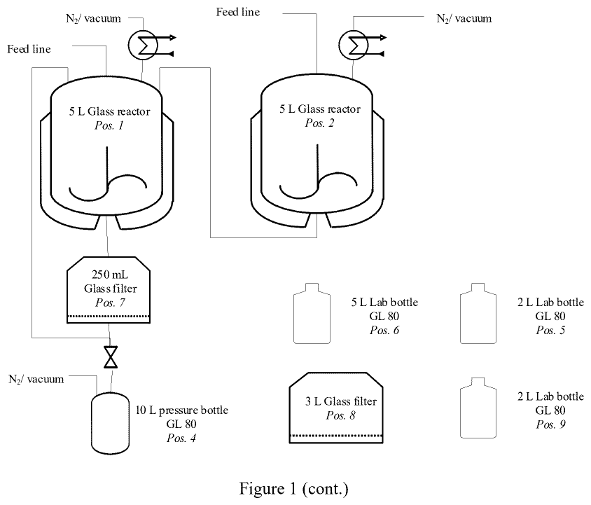

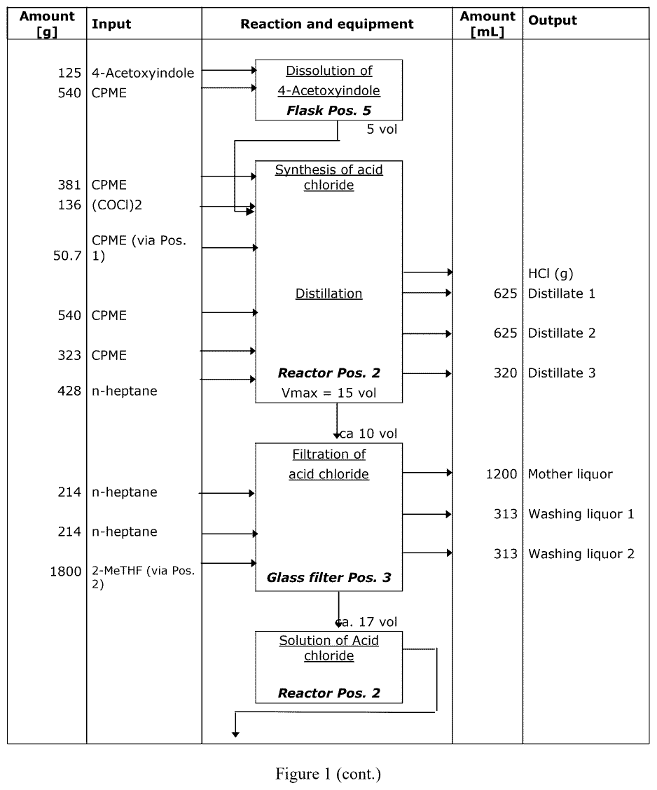

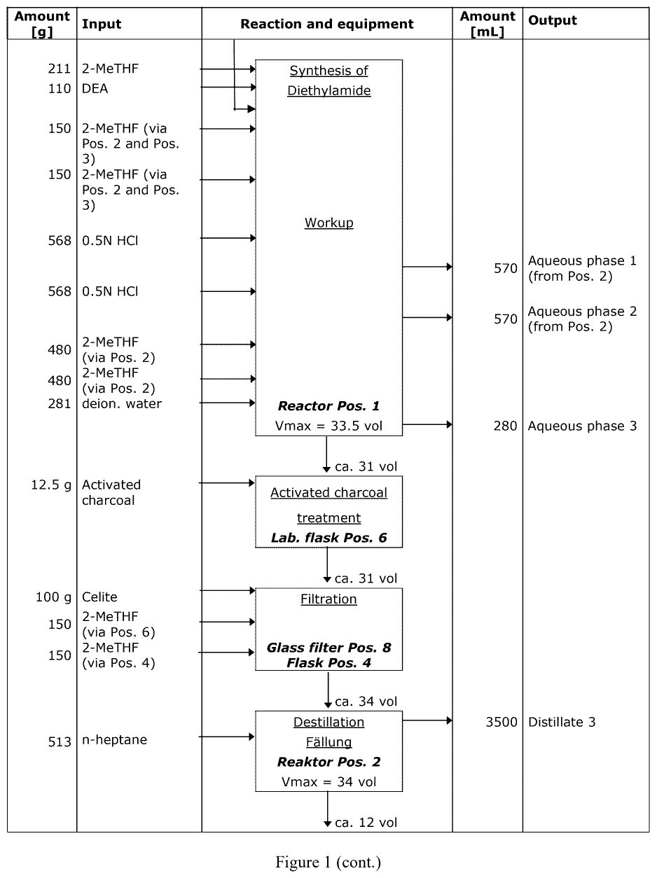

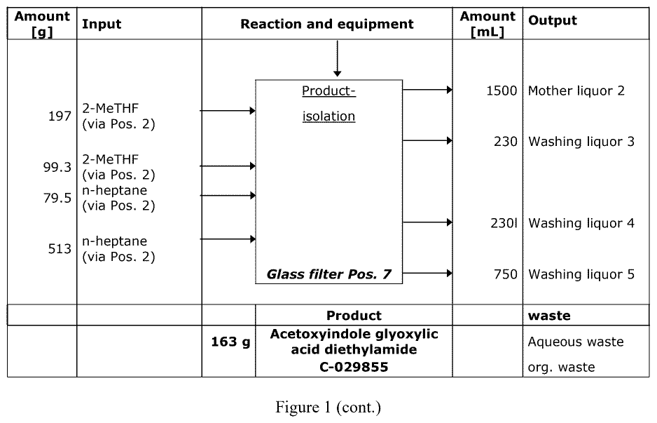

FIG. 1 describes the apparatus set up for step 1 of the examples, which describes the acid chloride formation step in the process for the preparation of acetoxyindole glyoxylic acid diethylamide and corresponding mass flow and mass balance chart, as well as the amide formation step in the process for the preparation of acetoxyindole glyoxylic acid diethylamide and corresponding mass flow and mass balance chart.

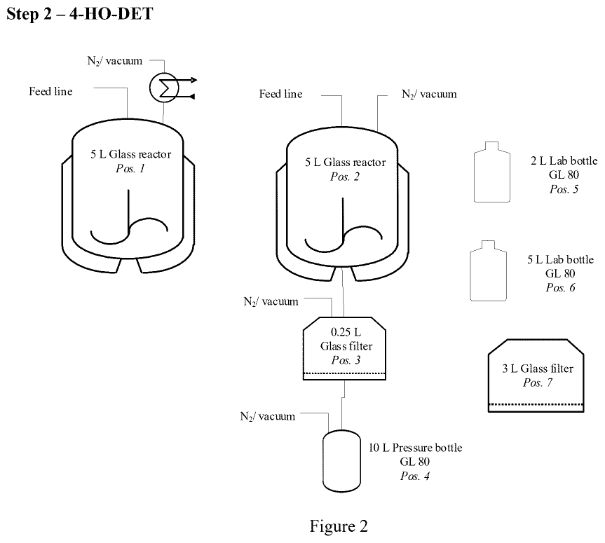

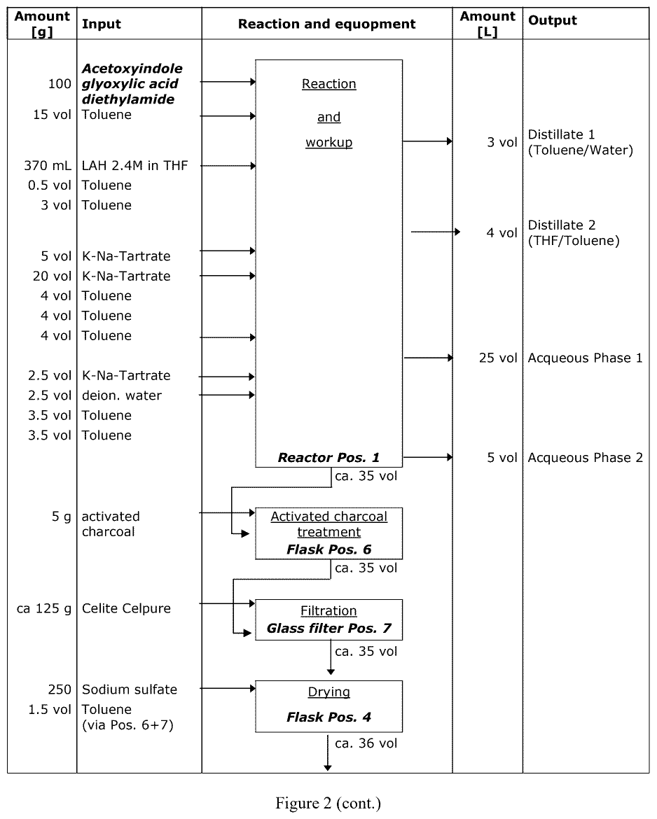

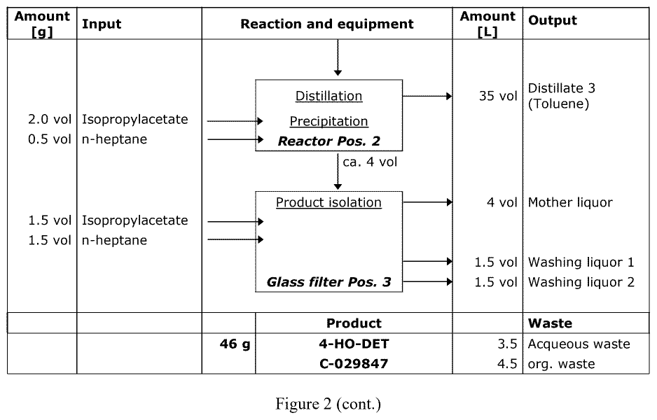

FIG. 2 describes the apparatus set up for step 2 of the examples, which describes the process for the preparation of 4-HO-DET and corresponding mass flow and mass balance chart.

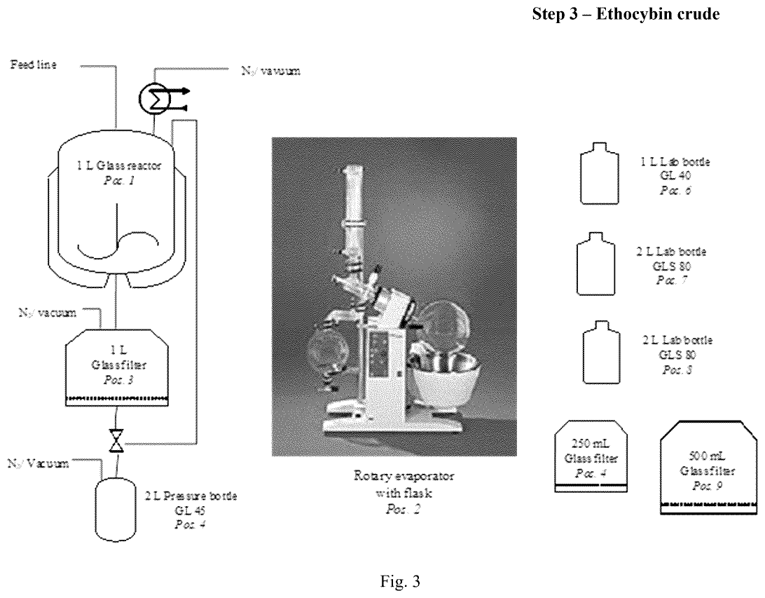

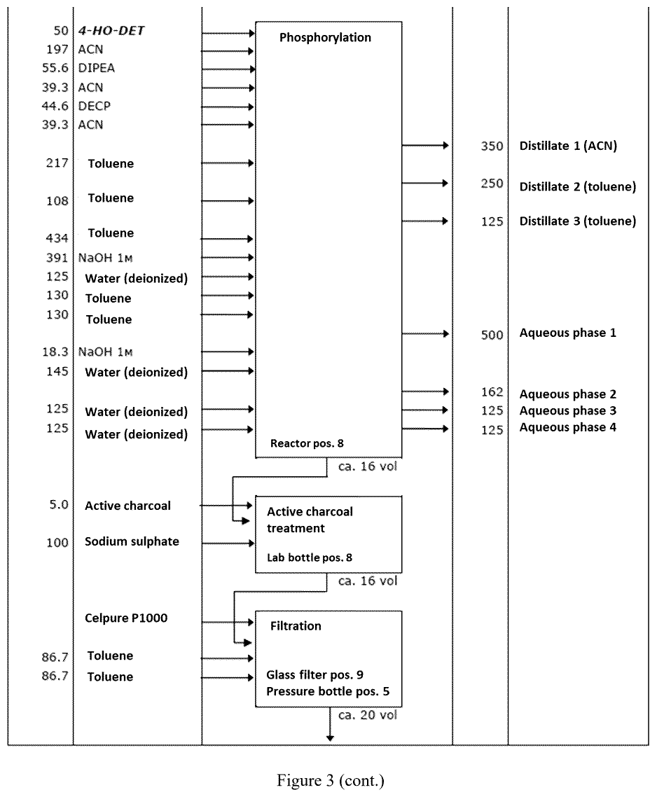

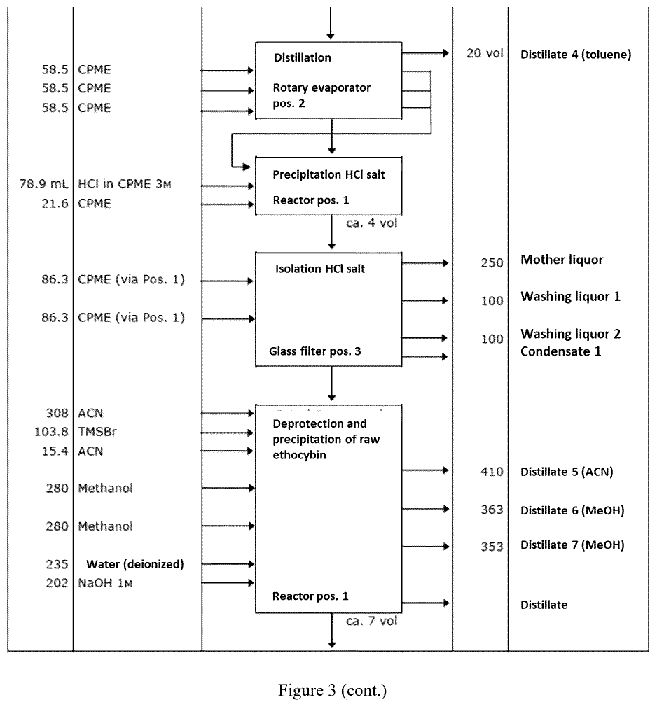

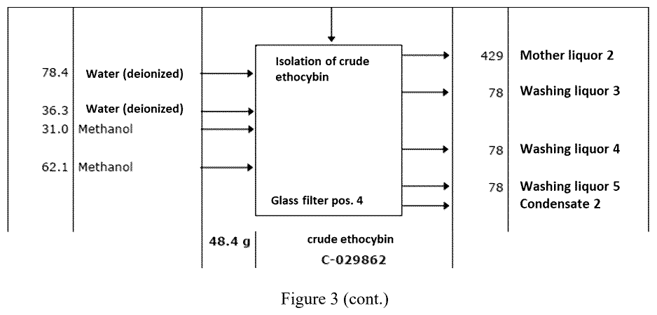

FIG. 3 describes the apparatus set up for step 3 of the examples, which describes the process for the preparation of the ethocybin crude product and corresponding mass flow and mass balance chart.

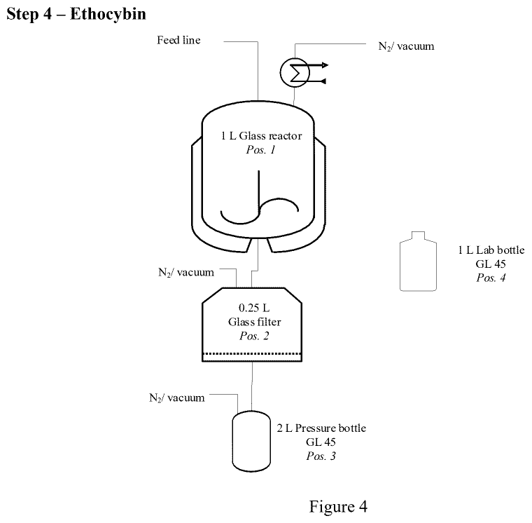

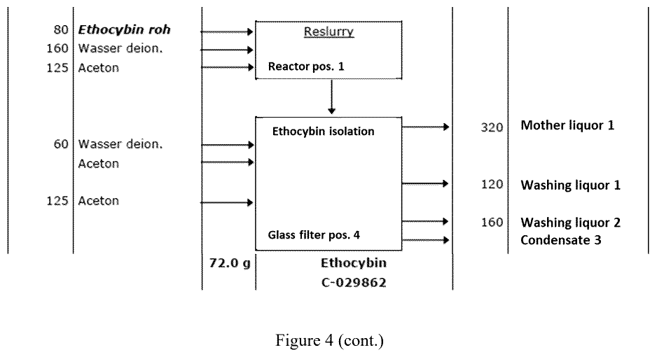

FIG. 4 describes the apparatus set up for step 4 of the examples, which describes the preparation of the final ethocybin product and corresponding mass flow and mass balance chart.

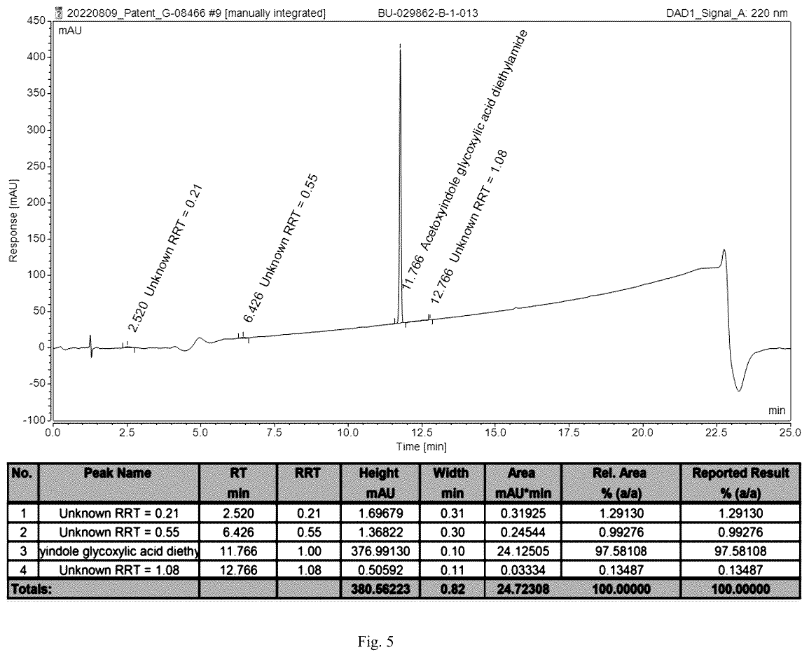

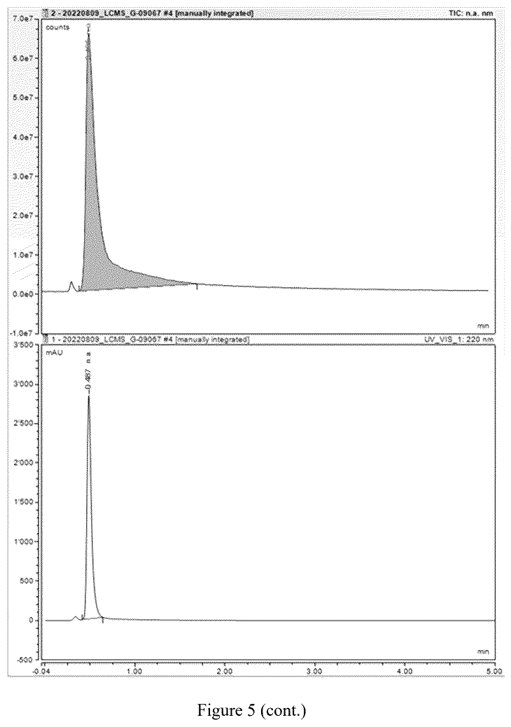

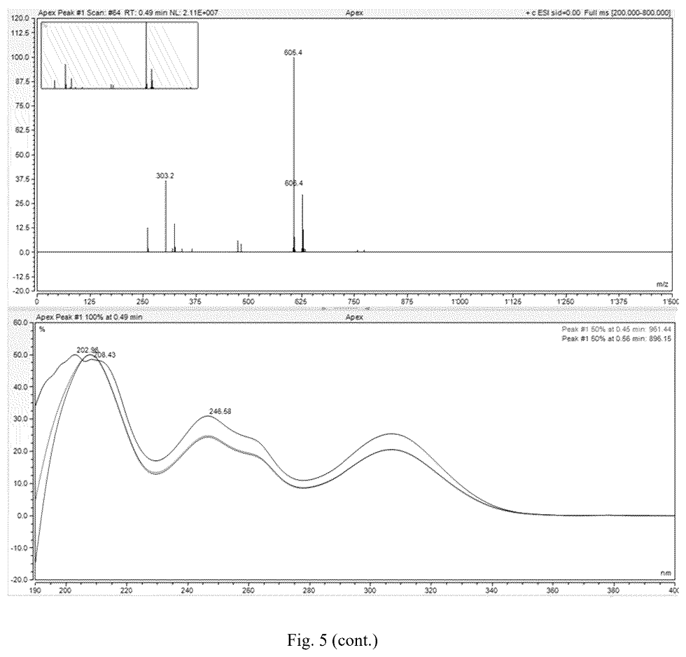

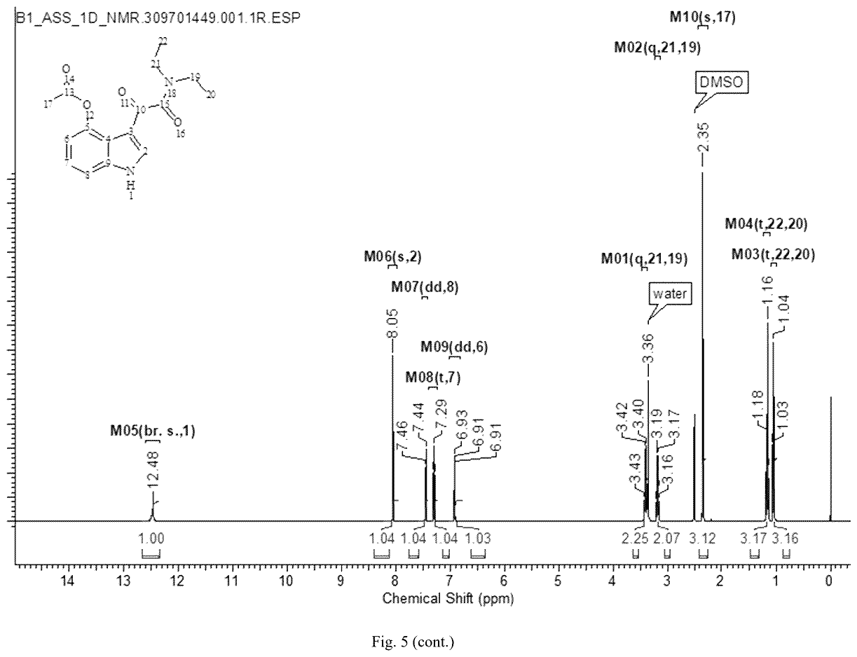

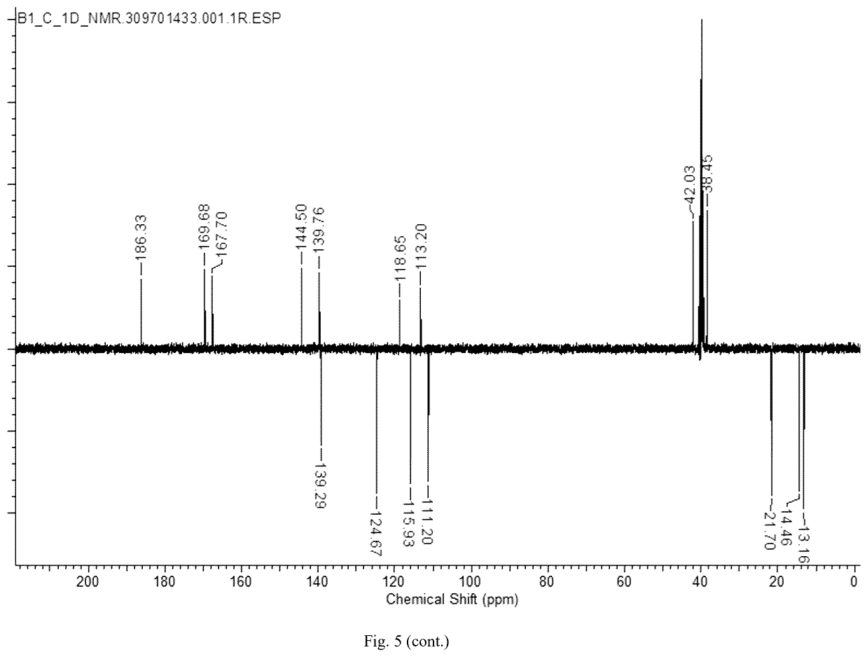

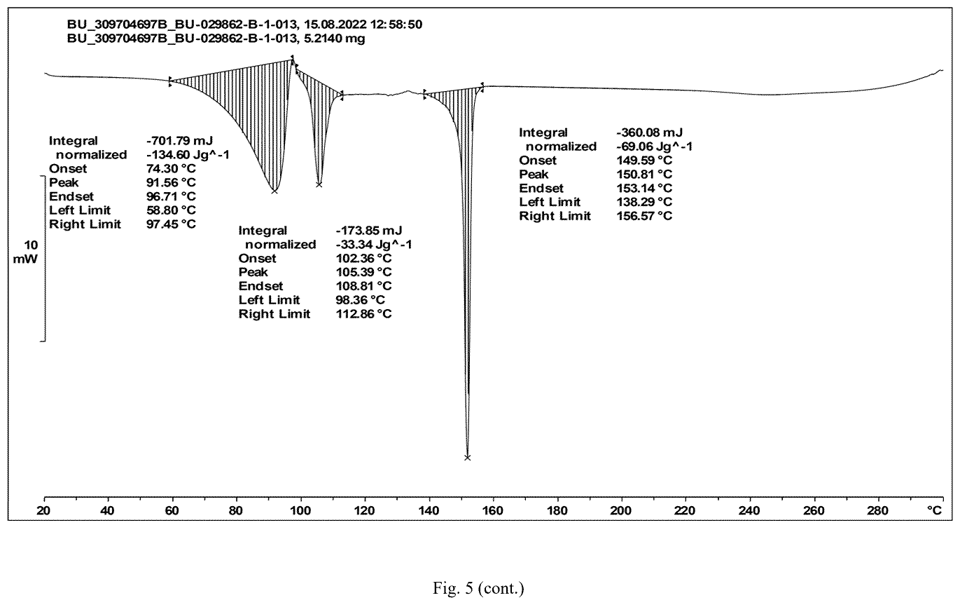

FIG. 5 shows analytical data for acetoxyindole glyoxylic acid diethylamide (the product of step 1 in the Examples), i.e., HPLC data (part 1), LC-MS data including UV-VIS spectrum (parts 2 and 3), ¹H and ¹³C NMR spectrum (parts 4 and 5, respectively), and DSC measurements (part 6).

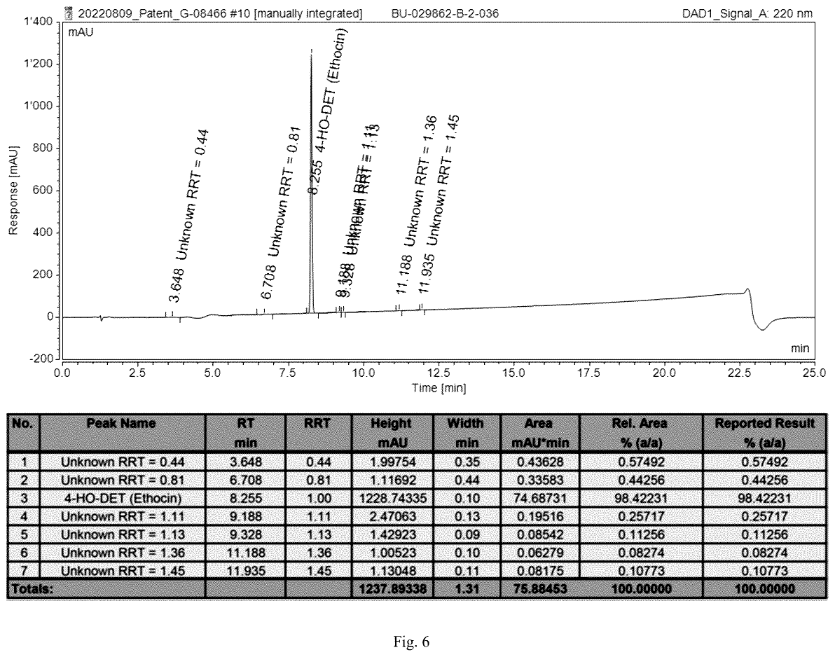

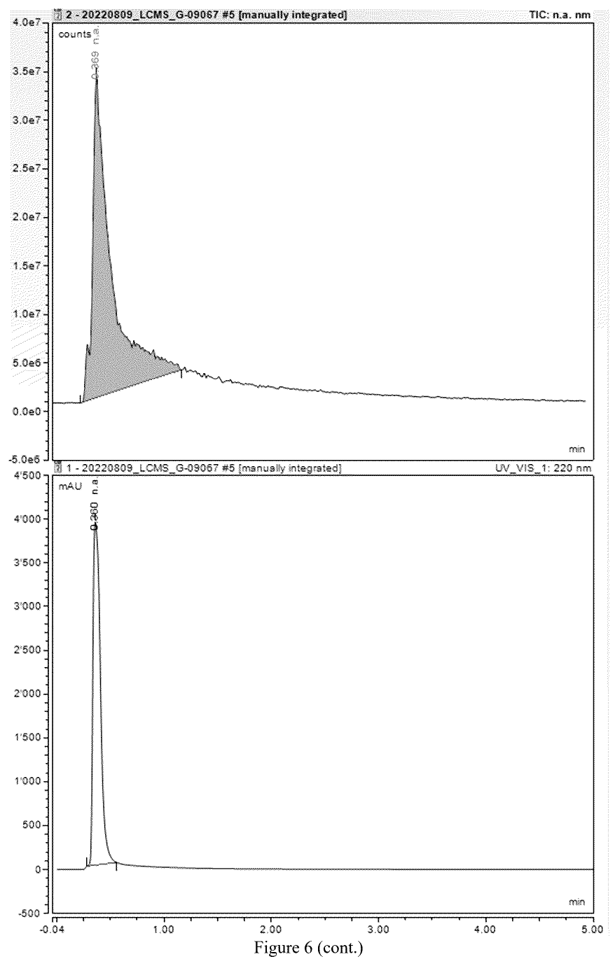

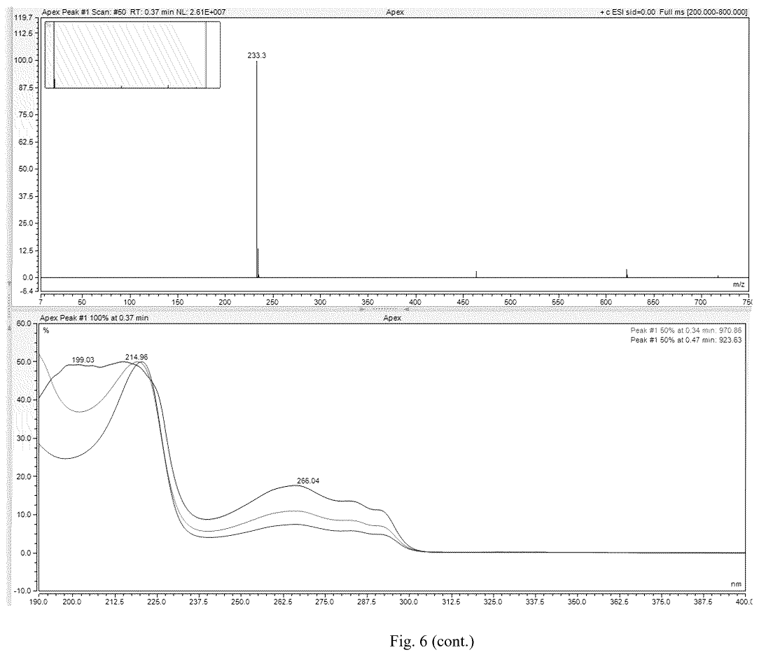

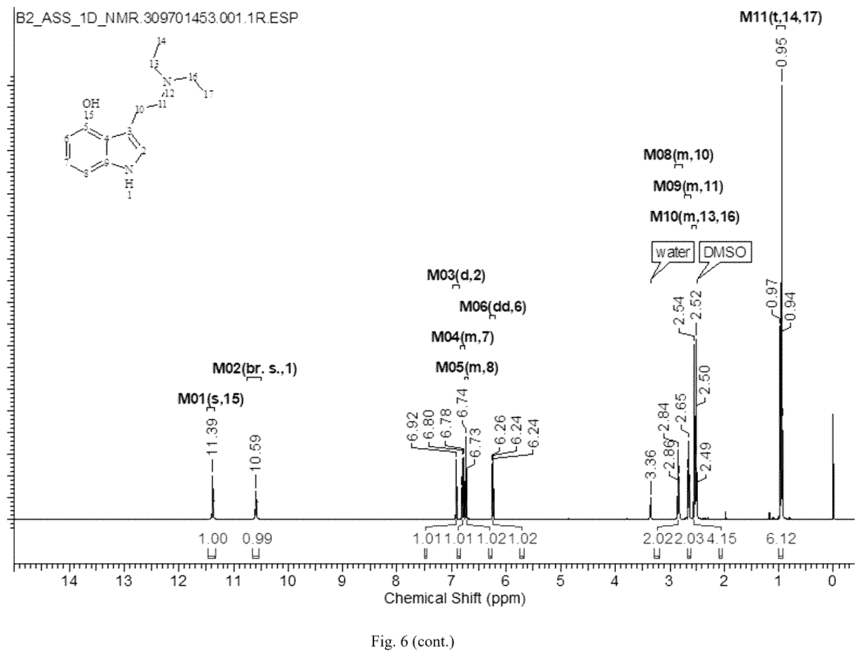

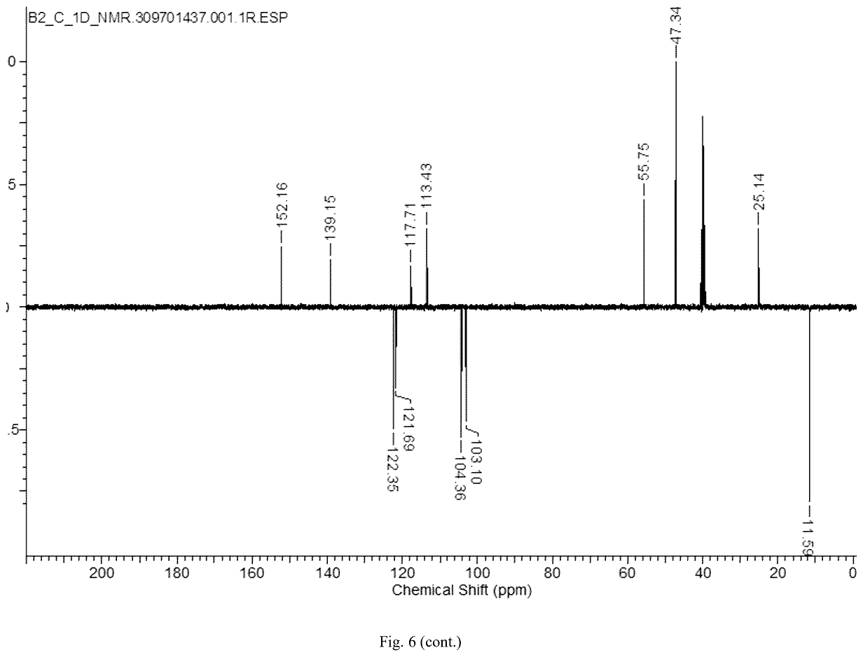

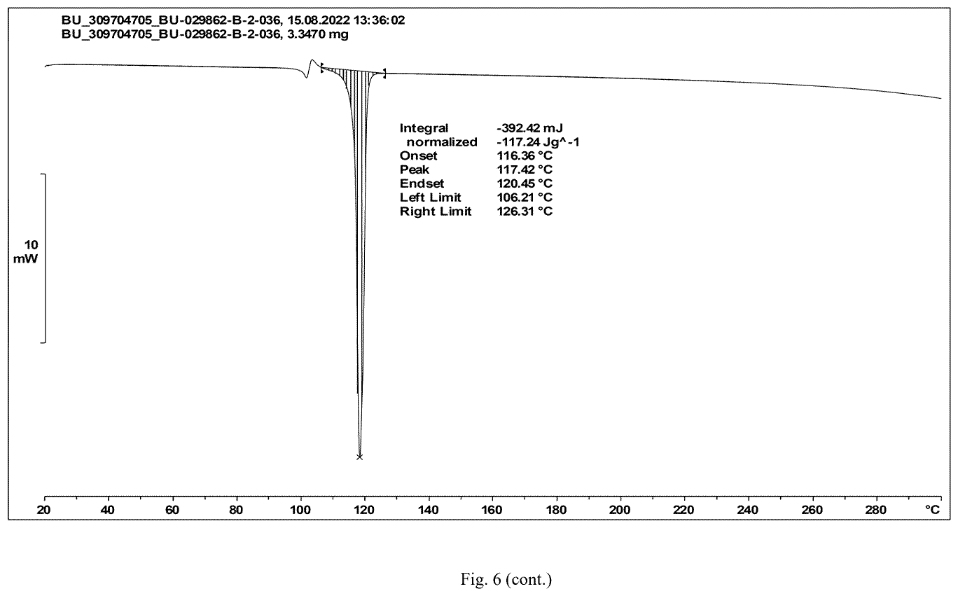

FIG. 6 shows analytical data for ethocin (4-OH-DET, the product of step 2 in the Examples), i.e., HPLC data (part 1), LC-MS data including UV-VIS spectrum (parts 2 and 3), ¹H and ¹³C NMR spectra (parts 4 and 5, respectively), and DSC measurements.

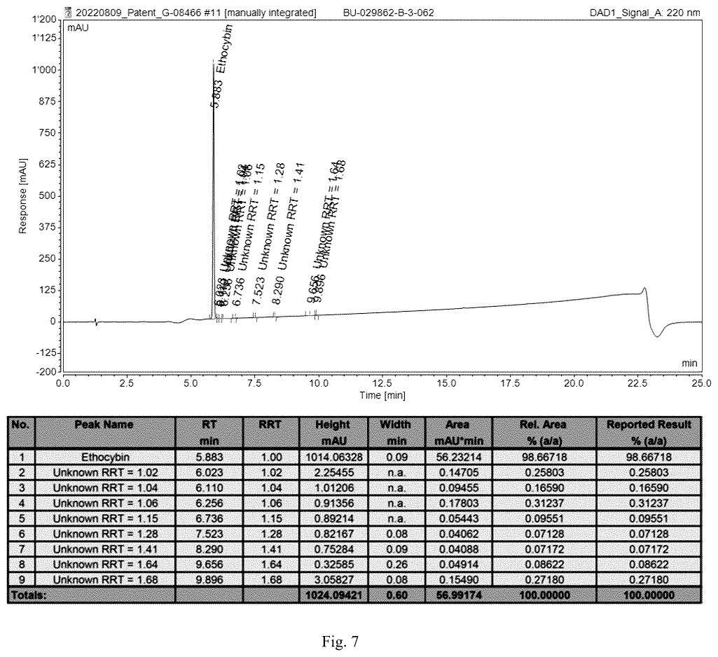

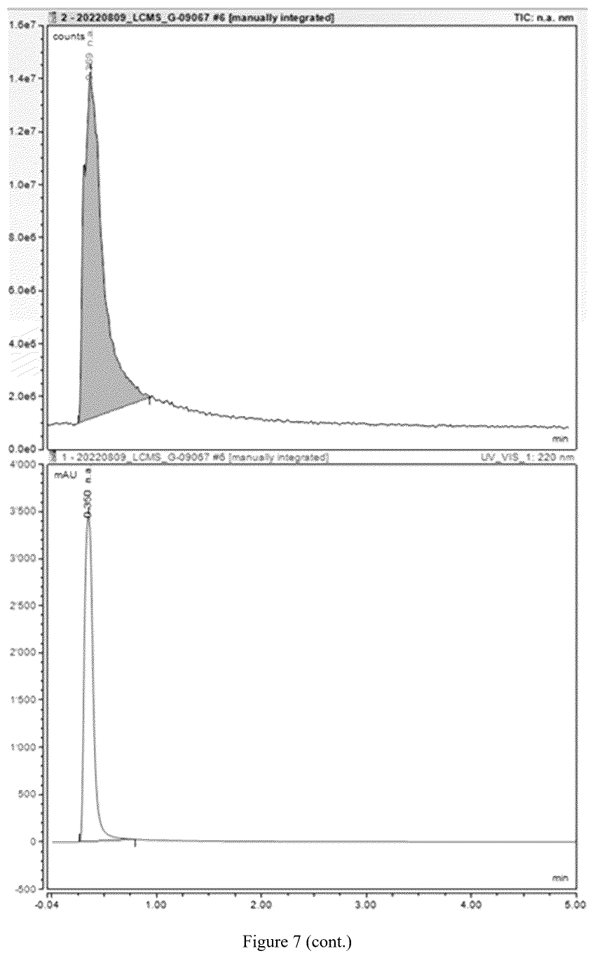

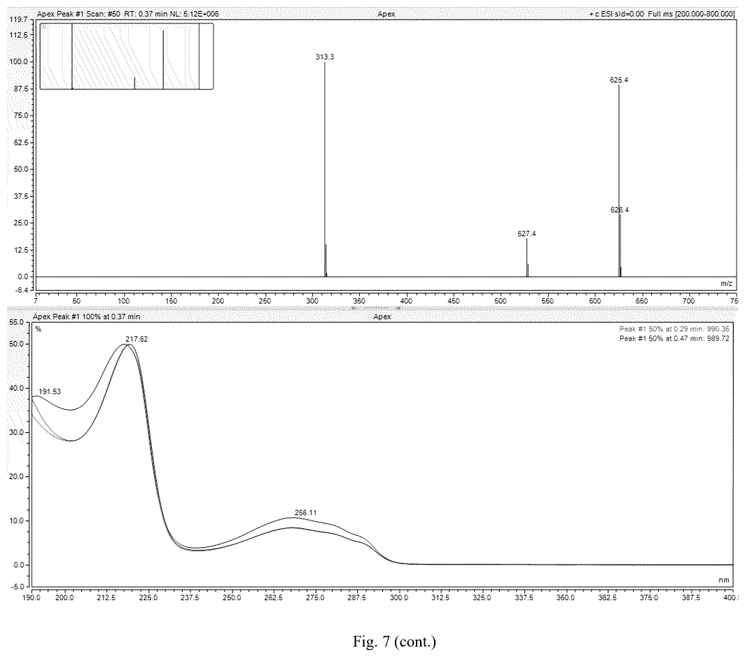

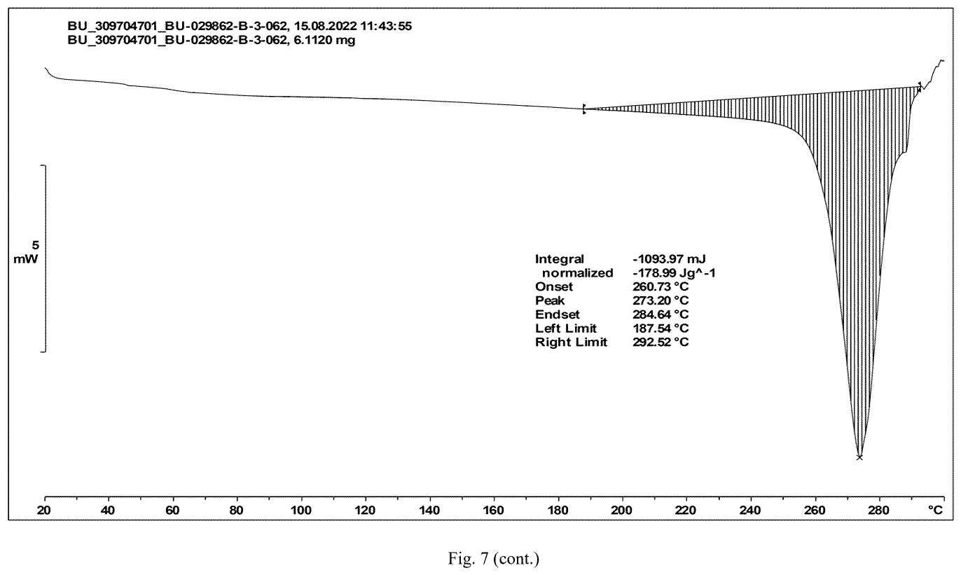

FIG. 7 shows analytical data for crude ethocybin (the product of step 3 in the Examples), i.e., HPLC data (part 1), LC-MS data including UV-VIS spectrum (parts 2 and 3), and DSC measurements (part 4).

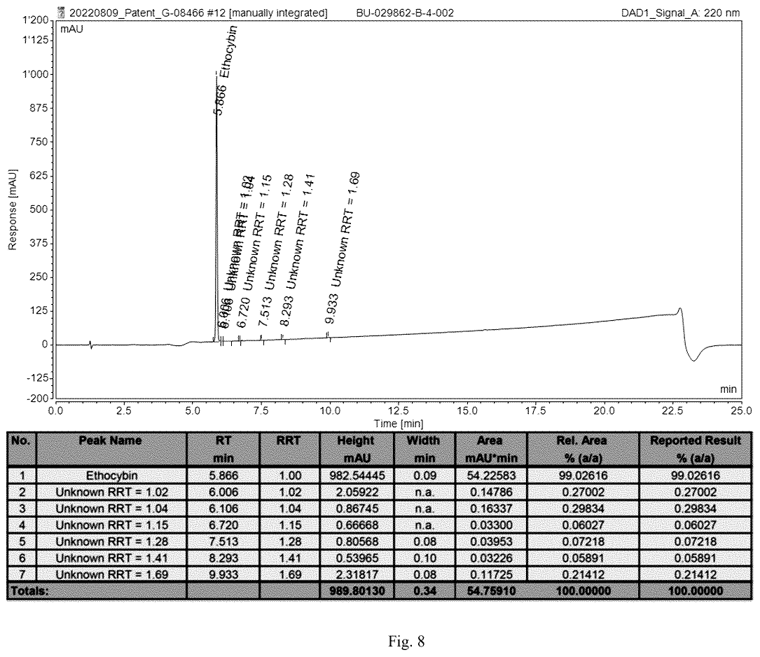

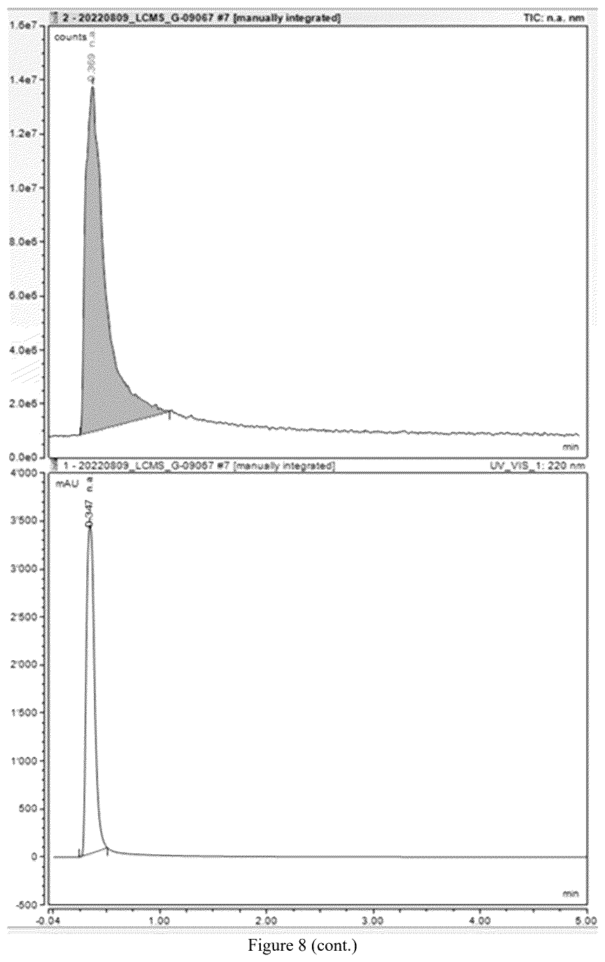

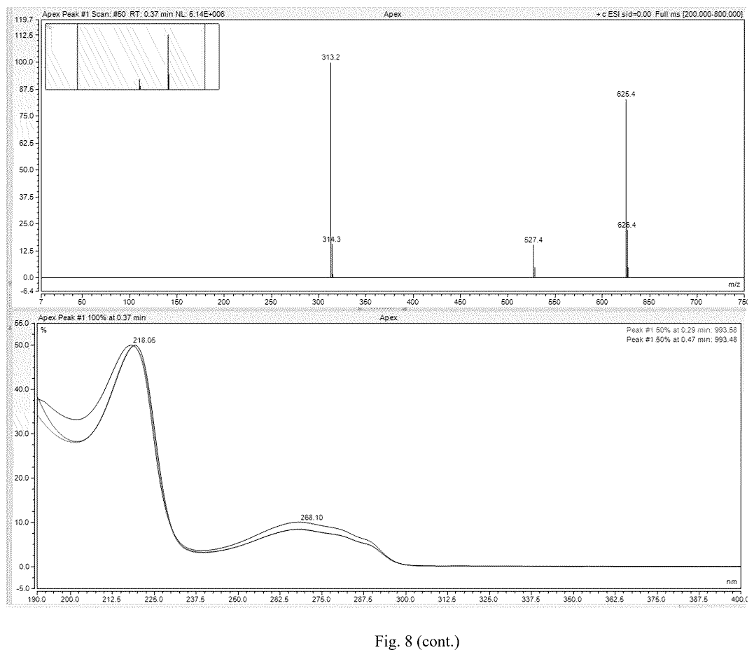

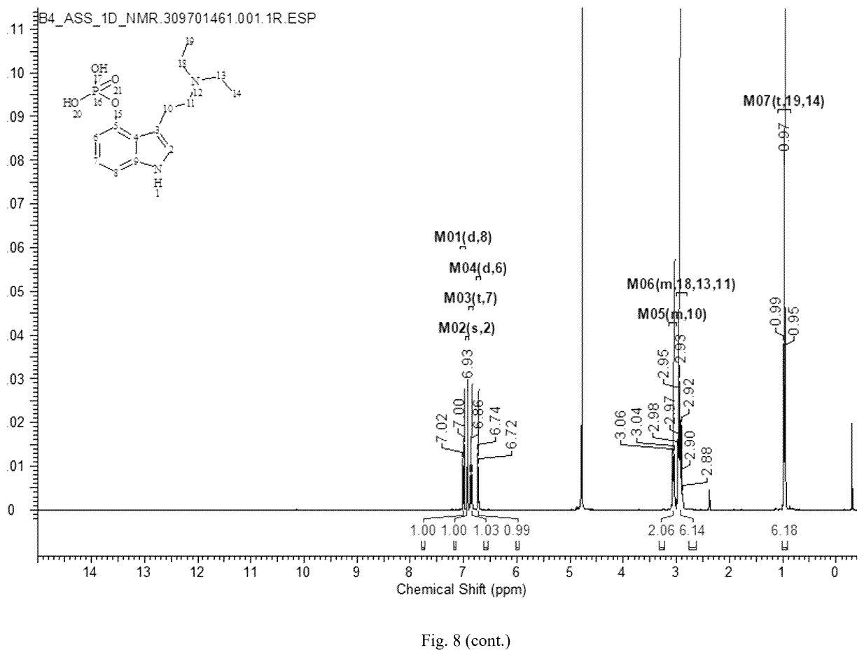

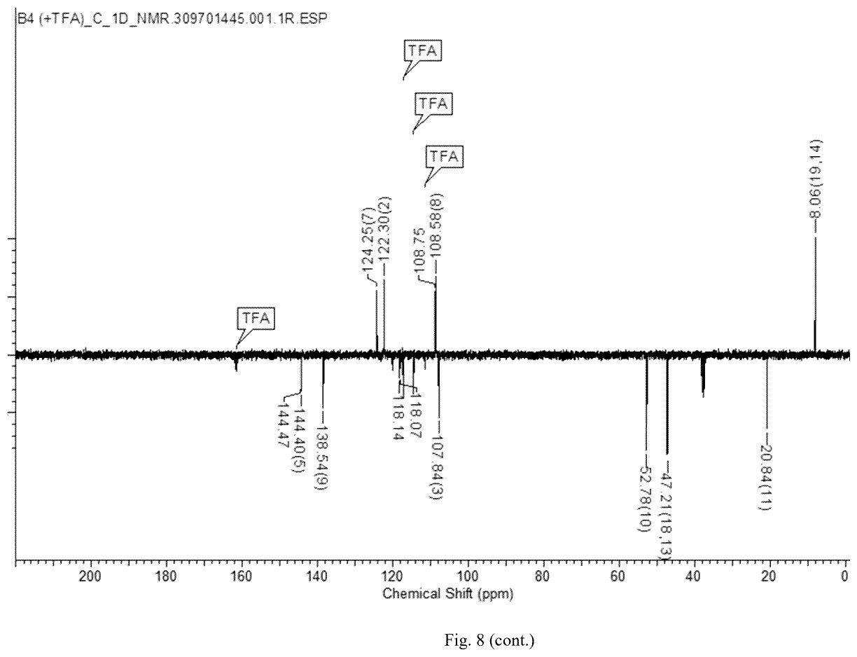

FIG. 8 shows analytical data for ethocybin (API/Drug Substance/Final Product), the product of step 4 in the Examples, i.e., HPLC data (part 1), LC-MS data including UV-VIS spectrum (parts 2 and 3), ¹H and ¹³C NMR spectra (parts 4 and 5, respectively), and DSC measurements.

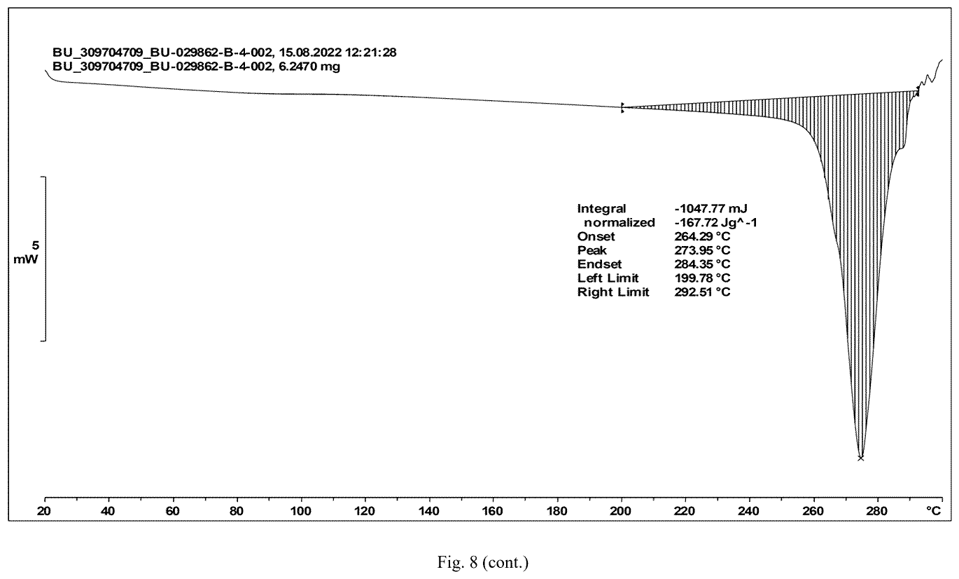

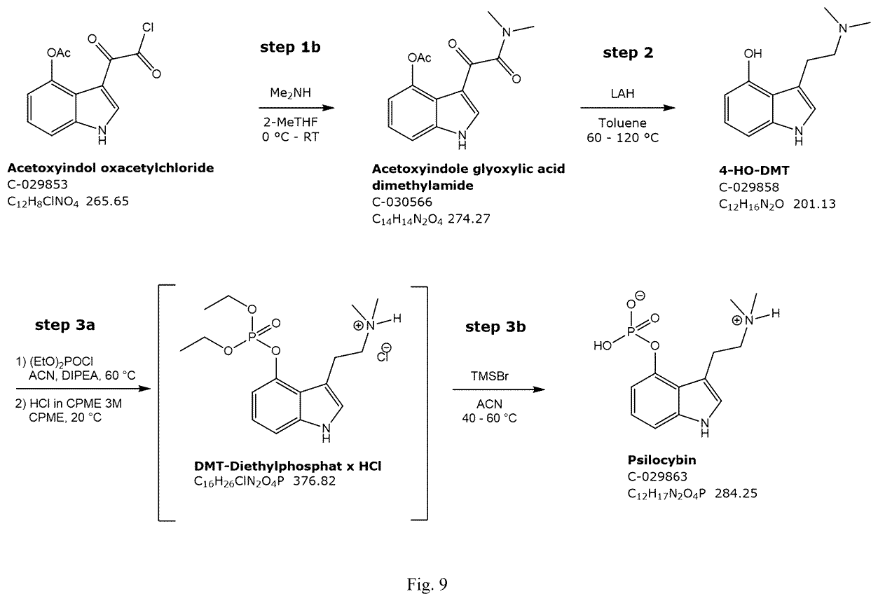

FIG. 9 shows exemplary reaction scheme leading to obtaining of psilocybin (the compound of formula (I), wherein both R are methyl.

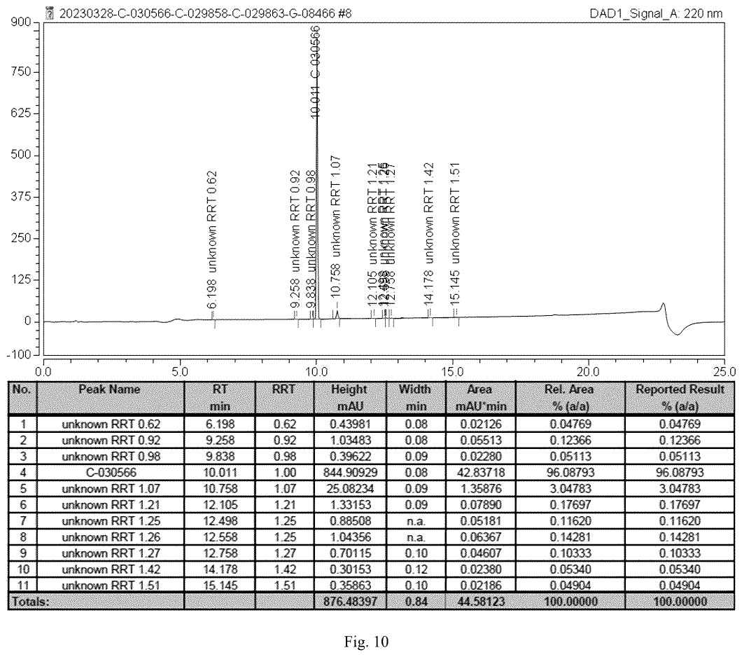

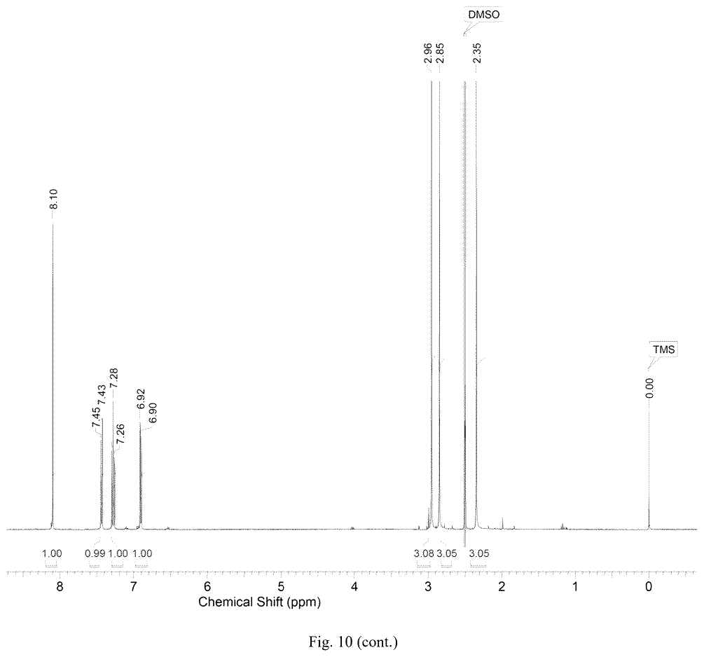

FIG. 10 shows HPLC data (part 1) and ¹H-NMR spectrum in d6-DMSO (part 2) for acetoxyindole glyoxylic acid dimethylamide.

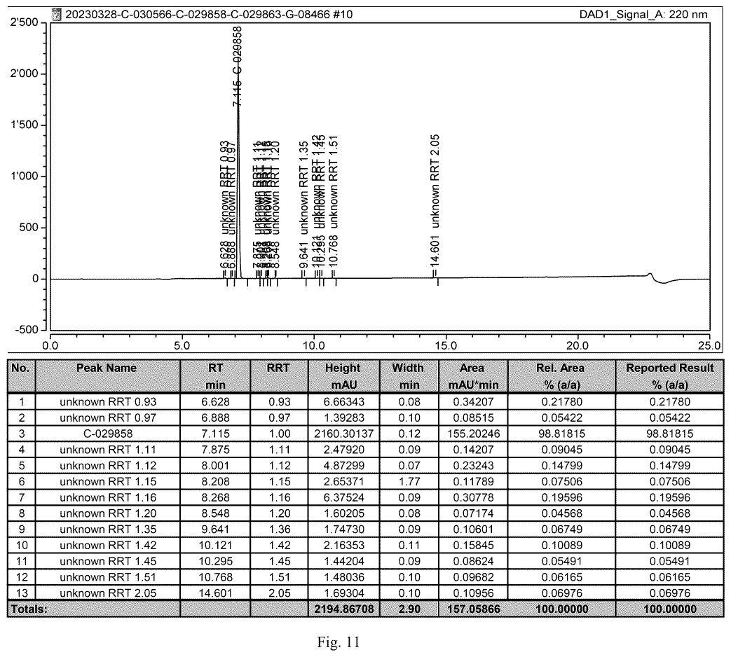

FIG. 11 shows HPLC data (part 1) and ¹H-NMR spectrum in d6-DMSO (part 2) for 4-Hydroxy-dimethyltrypthamine.

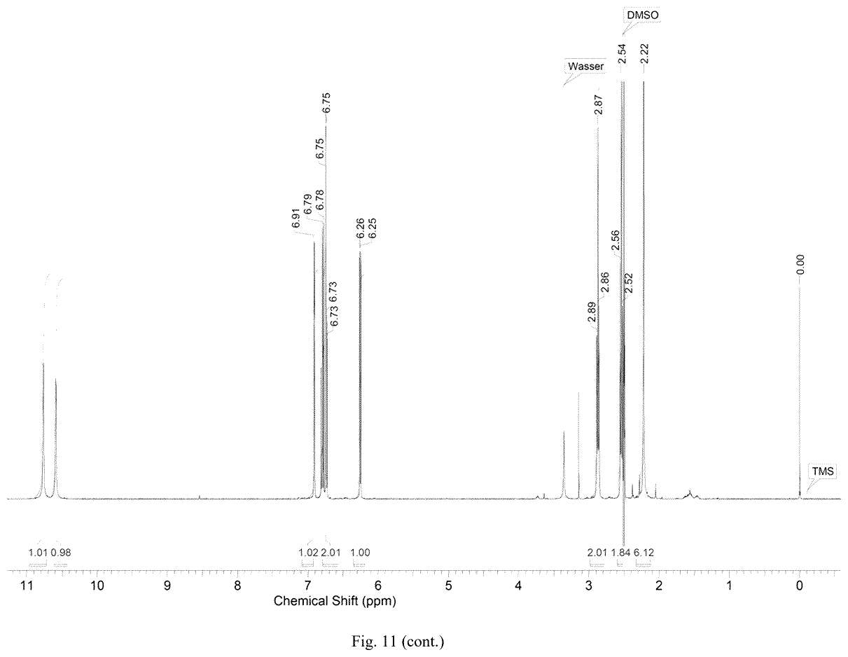

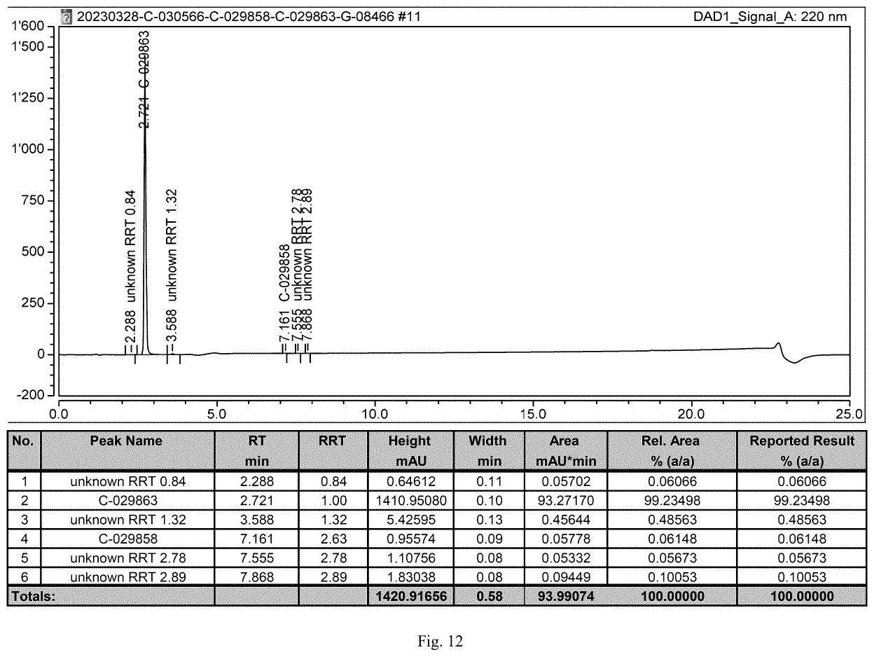

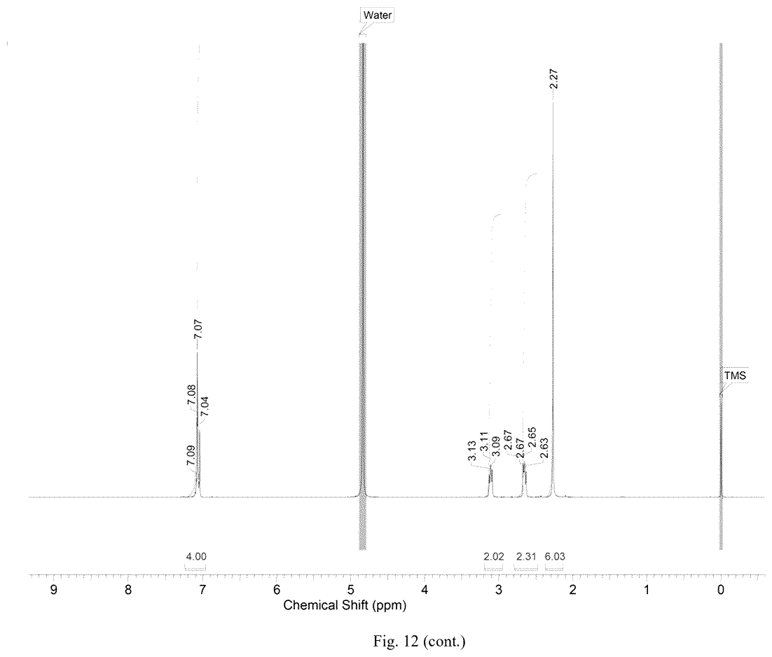

FIG. 12 shows HPLC data (part 1) and ¹H-NMR spectrum in D20 (part 2) for psilocybin.

### DETAILED DESCRIPTION OF THE INVENTION

Invention will be described in the following embodiments. It is to be understood that, unless indicated to the contrary, all possible combinations of disclosed steps and/or features are envisaged.

In one embodiment, the present invention relates to a method for preparing a compound of formula (I):

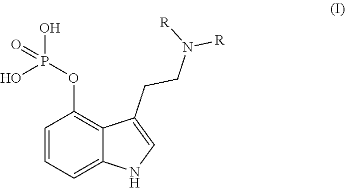

wherein each R is independently C₁₋₆ alkyl. Preferably, each R is independently methyl or ethyl. More preferably, each occurrence of R is ethyl.

The method of the present invention comprises the step of reacting the compound of formula (Id):

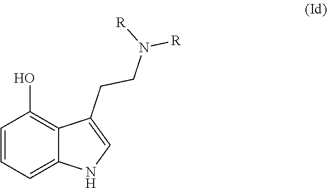

with (EtO)₂POCl (which can also be referred to as diethyl chlorophosphite or DECP).

Preferably, in the method of the present invention the w/w ratio of the compound of formula (Id) and (DECP) is between 0.8 and 1.2. More preferably, the w/w ratio of the compound of formula (Id) and (DECP) is between 1.0 and 1.2. Even more preferably the w/w ratio of the compound of formula (Id) and (DECP) is between 1.10 and 1.15. Even more preferably the w/w ratio of the compound of formula (Id) and (DECP) is about 1.12. Most preferably the w/w ratio of the compound of formula (Id) and (DECP) is 1.12.

As understood herein, the term “about” whenever referring to a number representing a ratio or a concentration is meant to be understood as preferably ±2%, more preferably as ±1%. Furthermore, as understood herein, the term “about” whenever referring to a temperature is meant to be understood as preferably ±1 °C, more preferably as ±0.5 °C.

A skilled person executing the step of reacting the compound of formula (Id) with DECP would be capable of arranging for an appropriate mixing of both components. According to the present inventors, it may be considered preferred to carry out the reaction in a way that avoids too much excess of DECP over (Id) at any given time. This may be achieved by adding (or dosing) DECP into (Id). Thus, it is preferred that DECP is added to (Id). It is further preferred that DECP is added to (Id) dropwise, preferably over the time of at least 10 minutes, more preferably over a time of between 10 and 30 minutes, even more preferably over the time of between 10 and 20 minutes.

According to the present inventors, the reaction of DECP with the compound of formula (Id) is preferably to be performed in a polar and aprotic solvent, for example in acetonitrile or DMF. Particularly preferred solvent for this reaction is acetonitrile. Thus, in a further embodiment of the invention the reaction of the compound of formula (Id) with DECP is performed in acetonitrile.

The use of solvents including acetonitrile or DMF that are characterized by a relatively high boiling point under normal conditions allows for flexible selection of the reaction temperature, as required by the reactants. According to the present inventors, the reaction of the compound of formula (Id) with DECP is preferably performed at a temperature of between 50 °C and 70 °C, more preferably at a temperature of between 55 °C and 65 °C, even more preferably at a temperature of between 57 °C and 63 °C, even more preferably at a temperature of about 60 °C, even more preferably at a temperature of 60 °C.

The reaction of the compound of formula (Id) with DECP is performed in the presence of a base. The present inventors have established that a preferred base is N-ethyl-diisopropylamine. However, this is not meant to be limiting and the skilled person would consider using other bases in order to establish their performance in the process of the present invention.

Preferably, according to the present invention, the reaction of the compound of formula (Id) with DECP is performed until a certain desired fraction of the compound of formula (Id) has been reacted. This is to be understood to be equivalent for a certain fraction of the compound of formula (Id) to have been consumed, irrespective of the reaction undergone by the compound. Preferably, the reaction is to be performed until at least 90%, at least 95% or 99% of the compound of formula (Id) has been reacted. More preferably, the reaction is to be performed until at least 99% of the compound of formula (Id) has been reacted. Preferably, the progress of the reaction is monitored by using LC-MS or GC-MS analysis on samples originating from the reaction mixture. Accordingly, the present inventors have established that preferably the reaction between DECP and the compound of formula (Id) is to be performed for a time of at least 150 minutes, until at least 99% of the compound of formula (Id) has been reacted. Afterwards, in the next step of the reaction of the compound of formula (Id) with DECP, the solvent is changed to toluene and aqueous NaOH solution is added thereto. As it will be apparent to the skilled person, the phase separation is expected to occur at this point. The skilled person tasked with preparing a suitable NaOH solution for this purpose would preferably not exceed NaOH concentration of 2.0 M. Preferably, in the aqueous phase that is being added NaOH is at the concentration of 0.5 M to 1.0 M, preferably at the concentration of about 0.75 M, more preferably at the concentration of 0.75 M. This step of the work up of the reaction mixture may also be referred to by the skilled person as a basic aqueous workup. The aqueous phase is separated from the toluene phase. The so obtained aqueous phase during the workup is preferably extracted twice with toluene and the toluene fractions (which according to the present inventors comprise the product of the reaction of the compound of formula (Id) with DECP) are combined. The so combined toluene phase is then washed with diluted NaOH aqueous solution (for example with 0.1 M NaOH solution, but other concentrations would be considered as being suitable by the skilled person as well) and subsequently washed twice with deionized water. As known to the skilled person, the aqueous workup would be completed by stirring the so obtained toluene phase with active carbon, drying the same over sodium sulphate and filtering the so obtained fraction upon Celite. The so worked-up toluene fraction is thereby ready for use in the next steps.

Upon completion of the reaction of the compound of formula (Id) with DECP, and upon performing toluene/aqueous NaOH workup as described hereinabove, the solvent is changed to cyclopentylmethylether (CPME). Subsequently, HCl in CPME is added to the reaction mixture, leading to precipitation of a crude product. Preferably, exchange of the solvent is performed through evaporation of the previous solvent with gradual addition of the target solvent, herein CPME. Preferred concentration of HCl in CPME does not exceed 5 M, more preferably is between 2 and 4 M. In a preferred embodiment, HCl in CPME is about 3M solution. In order to avoid undesired side-reaction, as it will be apparent to the skilled person, said solution of HCl in CPME is added dropwise over a certain period of time. Preferably, said solution of HCl in CPME is added dropwise over a time span of at least 10 minutes, preferably over a time span of at least 20 minutes. More preferably, said solution of HCl in CPME is added dropwise over a time span of between 20 and 40 minutes, even more preferably over a time span of between 25 and 35 minutes. As it is apparent to the skilled person, it is preferred to control the temperature during addition of HCl in CPME to the reaction mixture. Accordingly, in a preferred embodiment of the invention said solution is added at a temperature of between 15 °C and 25 °C, preferably at a temperature of between 17 °C and 23 °C, more preferably at a temperature of about 20 °C, even more preferably at a temperature of 20 °C.

According to the present inventors, upon addition of HCl in CPME a precipitate of the crude product of the reaction of the compound of formula (Id) with DECP is formed. The so obtained suspension is then preferably filtered under reduced pressure and the so obtained filtercake is washed with CPME, as apparent to the skilled person. The filtercake (which may also be referred as the crude product of the reaction of the compound of formula (Id) with DECP) is afterwards dried under reduced pressure.

Without being bound to the theory, it is noted that the reaction of the compound of formula (Id) with DECP results in a crude product of formula (Ie):

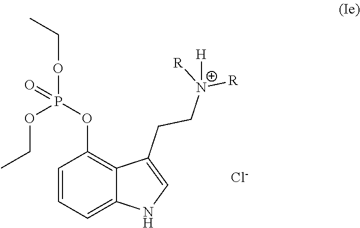

In formula (Ie), each R is as defined for formula (I). Thus, according to the present inventors, the crude product precipitated from CPME upon addition of HCl, as described hereinabove, comprises a compound of formula (Ie). Thus, in a preferred embodiment of the present invention, the reaction of the compound of formula (Id) with DECP results in a crude product of formula (Ie). In a further preferred embodiment of the present invention, the crude product precipitated from CPME upon addition of HCl comprises a compound of formula (Ie).

According to the present inventors, a compound of formula (If):

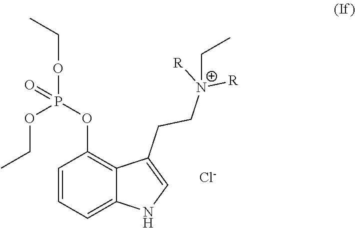

may be present in the reaction mixture. In formula (If), each R is as defined for formula (I). Without being bound by the theory, the present inventors postulate that this product is formed as a side product of the reaction of DECP with the compound of formula (Id), due to the presence of ethyl chloride, which according to the present inventors forms upon the reaction of the compound of formula (Id) with DECP. Further possible reasons for the occurrence of this side reaction include the presence of the ethyl chloride impurity in the preparation of DECP that has been used. According to the present inventors, the compound of formula (If) may also be formed in the reaction of alkylation of amine group in (Id) or in (Ie) (which is shown in the formulae hereinabove as protonated form) with DECP. Thus, the compound of formula (If) may according to the present inventors be present in the crude product of the compound of formula (Id) with DECP. The present inventors have demonstrated that the compound of formula (If) can be depleted in the reaction mixture, or substantially removed from the reaction mixture through the aqueous workup.

The present inventors have detected a compound of formula (Ig) of the reaction of the compound of formula (Id) and DECP:

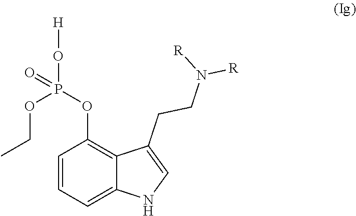

In formula (Ig), each R is as defined for formula (I). The present inventors postulate that the compound of formula (Ig) forms in the process of basic aqueous workup of said product. According to the present inventors, the compound of formula (Ig), if present in the reaction mixture, can be extracted during the basic aqueous workup and thus can be separated from the product. It has been however noted by the present inventors that formation of the compound of formula (Ig) may have a significant impact on the yield of the process.

The present inventors further postulate that the formation of the compound of formula (Ig) can preferably be suppressed (or reduced) by controlling the temperature, duration and pH of the reaction. More preferably, the formation of the compound of formula (Ig) can be suppressed (or reduced) by controlling the temperature. It is accordingly preferred that the addition of NaOH is performed at the temperature of 0 to 5 °C. Preferably, the inner temperature of the reactor is considered here.

Preferably, within the scope of the present invention, the product of the reaction of the compound of formula (Id) with DECP, as described hereinabove, is further reacted with TMSBr, resulting in the compound of formula (I). This reaction may also be referred to herein as deprotection reaction.

Accordingly, the present invention relates to a method for preparing a compound of formula (I):

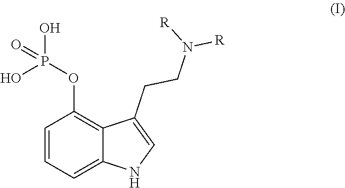

wherein each R is independently C₁₋₆ alkyl, preferably each R is independently methyl or ethyl,
wherein the method comprises the step of reacting the compound of formula (Id):

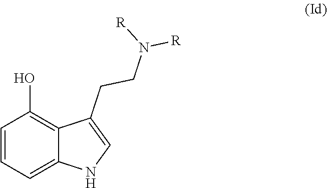

with (EtO)₂POCl (DECP), wherein the product of the reaction of the compound of formula (Id) with DECP is further reacted with TMSBr resulting in the compound of formula (I).

Preferably, the w/w ratio of TMSBr to the product of the reaction of DECP with the compound of formula (Id) is between 1.5 and 2.5. More preferably, the w/w ratio of TMSBr to the compound of formula (Id) is between 1.75 and 2.25. Even more preferably, the w/w ratio of TMSBr to the compound of formula (Id) is between 1.9 and 2.1. Even more preferably, the w/w ratio of TMSBr to the compound of formula (Id) is about 2.0. Even more preferably, the w/w ratio of TMSBr to the compound of formula (Id) is 2.0.

Preferably, the compound of formula (Id) is reacted with TMSBr in a polar and aprotic solvent. Accordingly, acetonitrile is a suitable solvent for this reaction. Thus preferably, the product of the reaction of DECP with (Id) is reacted with TMSBr in acetonitrile. As it is understood to the skilled person, this step would include dissolution of the crude product obtained in the previous step, which according to the present inventors may be the compound of formula (Ie), in acetonitrile. Accordingly, preferably within the scope of the present invention, the crude product of the reaction of the compound of formula (Id) is loaded together with the solvent, preferably acetonitrile, into a suitable reactor, as known to the skilled person.

Preferably, TMSBr is added to the product of the reaction of the compound (Id) with DECP, preferably TMSBr is added to the product of the reaction of the compound (Id) with DECP within 5 to 10 minutes, dissolved as described hereinabove, at a temperature of between 30 °C to 50 °C. More preferably TMSBr is added to the product of the reaction of the compound (Id) with DECP, dissolved as described hereinabove, at a temperature of between 35 °C and 45 °C. Even more preferably TMSBr is added to the product of the reaction of the compound (Id) with DECP, dissolved as described hereinabove, at a temperature of between 37 °C and 43 °C. Even more preferably TMSBr is added to the product of the reaction of the compound (Id) with DECP, dissolved as described hereinabove, at a temperature of about 40 °C. Even more preferably TMSBr is added to the product of the reaction of the compound (Id) with DECP, dissolved as described hereinabove, at a temperature of 40 °C

Preferably, the product of the reaction of the compound of formula (Id) with DECP is reacted with TMSBr at a temperature of between 50 °C and 70 °C. More preferably the product of the reaction of the compound of formula (Id) with DECP is reacted with TMSBr at a temperature of between 55 °C and 65 °C Even more preferably the product of the reaction of the compound of formula (Id) with DECP is reacted with TMSBr at a temperature of between 57 °C and 63 °C. Even more preferably the product of the reaction of the compound of formula (Id) with DECP is reacted with TMSBr at a temperature of about 60 °C. Even more preferably the product of the reaction of the compound of formula (Id) with DECP is reacted with TMSBr at a temperature of 60 °C.

The progress of the reaction of the product of the reaction of the compound of formula (Id) with DECP with TMSBr will be monitored. Preferably, according to the present invention, the reaction of the product of the reaction of the compound of formula (Id) with DECP with TMSBr is performed until a certain desired fraction of said product has been reacted. This is to be understood to be equivalent for a certain fraction of said product to have been consumed, irrespective of the reaction undergone by the compound. Preferably, the reaction is to be performed until at least 90%, at least 95% or 99% of said product has been reacted. More preferably, the reaction is to be performed until at least 99% of said product has been reacted. Preferably, the progress of the reaction is monitored by using LC-MS or GC-MS analysis on samples originating from the reaction mixture. Accordingly, the present inventors have established that preferably the reaction between the product of the reaction of DECP and the compound of formula (Id), which according to the present inventors may be the compound of formula (Ie), and TMSBr, is to be performed for a time of at least 150 minutes, until at least 99% of said product (which may be the compound of formula (Ie)) has been reacted.

Further encompassed by the present invention is an embodiment wherein TMSBr is replaced with TMSI (which, as it is apparent to the skilled person, can be in situ generated using TMSCl and iodide salt, e.g. KI). It is further apparent to the skilled person that in TMSBr (or TMSI/TMSCl, as applicable), the methyl group(s) can be replaced with other alkyl groups, for example selected from C₁₋₆ alkyl. However, most preferably, in the present invention, TMSBr is used, as described herein.

Preferably, upon completion of the reaction the solvent is changed to methanol and the reaction mixture is stirred in methanol. Preferably, said stirring is performed at a temperature of between 55 °C and 65 °C. More preferably, said stirring is performed at a temperature of between 57 °C and 63 °C. Even more preferably, said stirring is performed at a temperature of about 60 °C. Even more preferably, said stirring is performed at a temperature of 60 °C. Preferably said stirring is performed for a time of at least 20 minutes, more preferably for a time of between 25 and 35 minutes, even more preferably for a time of about 30 minutes, even more preferably for a time of 30 minutes.

Preferably, methanol is distilled away from the reaction mixture at this time point. It is to be understood that preferably at least 50%, more preferably at least 70% of methanol is distilled away. As it is understood by the skilled person, together with methanol certain by-products and side-products of the reaction may also be removed from the reaction mixture. The so obtained residue comprising residual methanol is further supplemented with methanol and stirred. Preferably, said stirring is performed at a temperature of between 55 °C and 65 °C. More preferably, said stirring is performed at a temperature of between 57 °C and 63 °C. Even more preferably, said stirring is performed at a temperature of about 60 °C. Even more preferably, said stirring is performed at a temperature of 60 °C. Preferably said stirring is performed for a time of at least 20 minutes, more preferably for a time of between 25 and 35 minutes, even more preferably for a time of about 30 minutes, even more preferably for a time of 40 minutes.

In one embodiment, the methanol can be removed completely and the so obtained solid residue upon removal of methanol is again dissolved in methanol, and the so obtained solution is preferably first incubated with active carbon, and subsequently filtered through Celite.

Afterwards, preferably the solvent is changed to water. Accordingly, most of methanol can be distilled away and the water is added. The pH of the so obtained solution is set to a value in a range of between pH=3.8 and pH=4.2. Preferably, setting of pH is done through the addition of aqueous solution of NaOH, as known to the skilled person. Preferably, according to the present inventors 1 M NaOH solution is to be used. Accordingly, the present inventors have used the w/w ration of 1 M NaOH solution to the original amount of the compound of formula (Id) of about 4.

Preferably, once the pH is set to a value in a range of between pH=3.8 and pH=4.2, the reaction mixture is concentrated under reduced pressure to reduce its volume.

Accordingly, and preferably, upon setting the pH to a value between 3.8 and 4.2, and preferably upon partially concentrating the solution, as described hereinabove, the final product of formula (I) precipitates from the so obtained aqueous solution thereof. Preferably, upon setting the pH to a value in a range of between pH=3.8 and pH=4.2, about four volumes, preferably four volumes (wherein said volumes are understood with respect to the volume of said obtained aqueous solution of the product of formula (I)) isopropanol are added to the aqueous solution and a distillation/concentration at jacket temperature of 70+/−5 °C is performed until approximately 3 volumes (as understood herein) of the solution in the reactor are left. To improve the yield of the process, the temperature of the process is to be controlled. Preferably, said aqueous solution is kept at a temperature of less than 20 °C. More preferably said aqueous solution is kept at a temperature of about 15 °C. Even more preferably said aqueous solution is kept at a temperature of 15 °C. It is to be preferably understood that cooling to jacket temperature of 15 °C as described herein is performed within 360 minutes.

Preferably, the so obtained precipitated product is filtered under reduced pressure and washed, preferably with deionized water, a deionized water/methanol mixture (preferably in a ratio of 1:1 v/v) and with methanol. The so obtained product is then dried under reduced pressure. Thereby, a crude preparation of the compound of formula (I) is obtained.

According to the present inventors, a compound of formula (Ih)

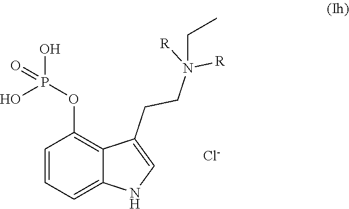

wherein each R is as defined for formula (I), may be present in the reaction product. Without being bound by the theory, the present inventors postulate that this product may be formed due to the presence of ethyl bromide in the reaction mixture, which according to the present inventors forms upon the reaction of the product of the reaction of the compound of formula (Id) with DECP (which may be the compound of formula (Ie)) with TMSBr. The present inventors have surprisingly found that the compound of formula (Ih) may be separated from the product of the deprotection reaction, i.e. from the compound of formula (I), in the process of crystallization of the compound of formula (I).

The present inventors have further detected a compound of formula (Ii) formed in the reaction of the product of the reaction between the compound of formula (Id) and DECP and TMSBr:

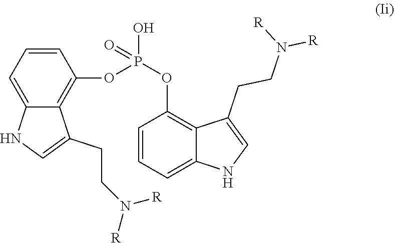

wherein each R is as defined for formula (I). The present inventors have surprisingly found that the compound of formula (Ii) may be separated from the product of the deprotection reaction, i.e. from the compound of formula (I), in the process of crystallization of the compound of formula (I).

Accordingly, the present invention further relates to an embodiment, wherein the so obtained crude product according to formula (I), which according to the present inventors may comprise further impurities including the compound of formula (Ih) and/or the compound of formula (II), is recrystallized or re-slurried, preferably re-slurried from water/acetone. Accordingly, the crude product of formula (I) of the present invention as described herein is slurried in water/acetone and refluxed at a temperature of 60 °C. Afterwards, the recrystallization/re-slurry mixture is allowed to cool to the temperature of about 20 °C, and formed precipitate is filtered out, washed with water/acetone (1:1) and with acetone, and dried under vacuum.

In one embodiment of the present invention, the method of the present invention comprises the step of reducing a compound of formula (Ic):

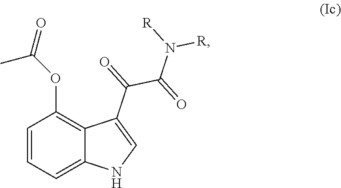

wherein each R is as defined for formula (I), resulting in the compound of formula (Id).

Several reducing agents suitable for reduction of the keto group and the amide group can be used in this embodiment of the method of the present invention. Accordingly, in one embodiment, LAH (also referred to as LiAlH₄) is used in the reduction reaction. Preferably, in this specific embodiment, the reduction is performed in toluene, dioxane or CPME. More preferably, said reduction is performed in toluene, preferably under reflux.

In the reaction of ketoamide (Ic) with LAH, in the first step said compound of formula (Ic) is loaded together with toluene into the reactor and any residual water is removed by performing an azeotropic distillation of toluene/water. Once the distillation is complete and the reaction mixture is cooled to a temperature of between 55 °C and 65 °C, preferably to a temperature of about 60 °C, more preferably to a temperature of 60 °C, the solution of LAH in THF (preferably 2.0M-3.0 M solution, in particular 2.4 M solution) is added. As known to the skilled person, during the addition of LAH both heat and gaseous hydrogen would be liberated from the reaction mixture. It is thus apparent to the skilled person that care should be taken in the process, for example by controlled dosing of the addition of LAH, and/or by including a light flow of a gas, for example nitrogen, in order to avoid increased concentration of hydrogen.

The reaction of the compound of formula (Ic) with the reducing agent is preferably performed until a certain desired fraction of said compound has been reacted. This is to be understood to be equivalent for a certain fraction of said compound to have been consumed, irrespective of the reaction undergone by the compound. Preferably, the reaction is to be performed until at least 90%, at least 95% or 99% of said compound has been reacted. More preferably, the reaction is to be performed until at least 99% of said compound has been reacted. Preferably, the progress of the reaction is monitored by using LC-MS or GC-MS analysis on samples originating from the reaction mixture.

Once the certain desired fraction of the compound of formula (Ic) has been consumed, as described hereinabove, preferably additional volume of toluene is loaded into the reactor, the reaction mixture is further concentrated. Then saturated solution of sodium-potassium tartrate is added. The so obtained composition is stirred, preferably at a temperature of about 40 °C, and the organic phase is separated from the aqueous phase. The aqueous phase is washed with toluene (preferably several times, for example four times) so that it can be extracted. The so obtained toluene phases are combined, washed with half-saturated sodium-potassium tartrate solution, incubated with active carbon, dried over sodium sulphate and/or filtered through Celite.

Thereafter the product is preferably crystallized from isopropylacetate/n-heptane. Accordingly, the organic phase is concentrated and isopropylacetate/n-heptane is added. Crystallization is induced by inoculation whereby the temperature is first reduced to 0 °C, and then to −30 °C. The crystallized compound of formula (Id) is separated by filtration under reduced pressure and the obtained filtrate is then dried under vacuum.

According to the present inventors, a compound of formula (Ij):

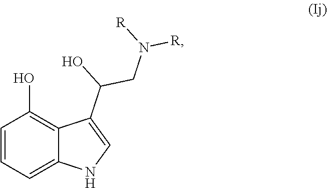

wherein each R is as defined for formula (I), may be formed during the reduction of the compound of formula (Ic). The present inventors have found however that surprisingly the compound of formula (Ij) can be separated from the compound of formula (Id) in the process of crystallization from isopropylacetate/n-heptane. It has further been postulated by the present invention that this compound, as an intermediate of the reduction, needs higher temperatures to be further reduced/converted. Accordingly and preferably, the temperatures obtainable by boiling toluene, dioxane, or CPME, are required. Said temperatures correspond to the boiling points of toluene, dioxane or CPME, respectively

In another specific embodiment of the present invention, the compound of formula (Ic) is reduced by using NaBH₄.

In another embodiment, the present invention relates to a method of the present invention, wherein the method further comprises the step of reacting a compound of formula (Ia):

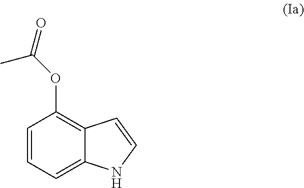

with (COCl)₂, followed by the reaction of the resulting product with R₂NH. Each R in R₂NH is as defined for formula (I).

Preferably, the reaction is to be performed in CPME as a solvent, wherein both oxalyl chloride and the compound of formula (Ia) should be dissolved. In this reaction, preferably excess of oxalyl chloride over the compound of formula (Ia) is to be used, for example at least 1.1 molar ratio, at least 1.5 molar ratio or at least 2.0 molar ratio of oxalyl chloride to the compound of formula (Ia). Particularly preferred is the use of molar ratio of about 1.5, more preferably of 1.5, of oxalyl chloride to the compound of formula (Ia). Preferably, the excess of oxalyl chloride is to be provided over the entire duration of the reaction. Accordingly, it is preferred that the solution of the compound of formula (Ia) is added, for example dropwise, to the solution of oxalyl chloride.

The temperature of the reaction is preferably to be controlled, and preferably the reaction between oxalyl chloride and the compound of formula (Ia) is to be performed at a temperature of not more than 20 °C, preferably at a temperature of between 0 °C and 20 °C, more preferably at a temperature of between 0 °C and 10 °C.

The progress of the reaction is preferably controlled by monitoring the compound of formula (Ia). Accordingly, the reaction between oxalyl chloride and the compound of formula (Ia) is preferably performed until a certain desired fraction of said compound has been reacted. This is to be understood to be equivalent for a certain fraction of said compound to have been consumed, irrespective of the reaction undergone by the compound. Preferably, the reaction is to be performed until at least 90%, at least 95% or 99% of said compound has been reacted. More preferably, the reaction is to be performed until at least 99% of said compound has been reacted. Preferably, the progress of the reaction is monitored by using LC-MS or GC-MS analysis on samples originating from the reaction mixture.

Preferably, once certain desired fraction of the compound of formula (Ia) has been reacted, as described hereinabove, the reaction mixture is diluted with the solvent, preferably with CPME, and said solvent is distilled out of the reaction mixture, removing oxalyl chloride at the same time.

Preferably, n-heptane is added to the reaction mixture, preferably at a temperature of not more than 20 °C, preferably at a temperature of between 0 °C and 20 °C, more preferably at a temperature of between 0 °C and 10 °C, even more preferably at a temperature of about 0 °C, even more preferably at a temperature of 0 °C. Consequently, the product of the reaction of the compound of formula (Ia) with oxalyl chloride precipitates and is separated by filtration and then washed with n-heptane. The so obtained filtrate is dried by exposure to a flow of nitrogen.

According to the present inventors, the reaction of the compound of formula (Ia) with oxalyl chloride results in a compound of formula (Ib):

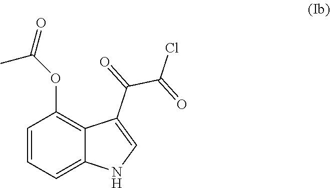 [SMILES: CC(=O)Oc1cccc2[nH]cc(C(=O)C(=O)Cl)c12]

As understood herein, the product of the reaction of the compound of formula (Ia) with oxalyl chloride, which according to the present inventors may be the compound of formula (Ib), is to be reacted with the amine of formula R₂NH (wherein each R is as defined for formula (I)) to yield a compound of formula (Ic).

Preferably, said reaction is to be performed in 2-MeTHF. Accordingly, the product of the reaction of the compound of formula (Ia) with oxalyl chloride upon filtration and drying under nitrogen can be directly dissolved in 2-MeTHF while still placed in the filtration funnel. Both substrates dissolved in said solvent are to be stirred together, monitoring the progress of the reaction. It is preferred that the product of the reaction of the compound of formula (Ia) with oxalyl chloride is added to the amine at a temperature of between-10 to 5 °C. Once a certain fraction of the product of the reaction of the compound of formula (Ia) with oxalyl chloride is reacted (for example at least 90%, at least 95%, or at least 99%), the reaction is stopped by addition of 1N HCl aqueous solution into the reaction mixture. The phase separation follows, and the separated organic phase is washed with aqueous HCl solution, for example with 0.5 M HCl solution. The so generated aqueous phases are then washed with 2-MeTHF, and combined organic phase is washed with water, incubated with active carbon and filtered through Celite.

The so obtained solution is concentrated and the crystallization of the product is initiated, whereby the temperature is decreased to −20 °C. The precipitated product of formula (Ic) is filtered, washed with 2-MeTHF, 2-MeTHF/n-heptane (preferably 1:1 v/v) and n-heptane, and dried under vacuum.

It is to be understood that in the embodiments of the present invention wherein the compound of formula (Id) is reacted with DECP, it is recognizable to the skilled person that DECP can be replaced with a compound of formula (R′O)₂POCl, wherein each R′ is independently selected from C₁₋₆ alkyl, —CH₂-aryl or —CH₂-heteroaryl, preferably wherein each R′ is independently selected from C₁₋₆ alkyl and —CH₂-aryl, more preferably wherein each R′ is independently C₁₋₆ alkyl. Accordingly, within the scope of the present invention, in the method of the present invention, said compound of formula (Id) can be reacted with a compound of formula (R′O)₂POCl, wherein each R′ is independently selected from C₁₋₆ alkyl, —CH₂-aryl or —CH₂-heteroaryl, preferably independently selected from C₁₋₆ alkyl and —CH₂-aryl, more preferably wherein each R′ is independently C₁₋₆ alkyl. Particularly suitable C₁₋₆ alkyl groups are ethyl and tert-butyl. Particularly suitable —CH₂-aryl is benzyl.

As used herein, the term “alkyl” refers to a monovalent saturated acyclic (i.e., non-cyclic) hydrocarbon group which may be linear or branched. Accordingly, an “alkyl” group does not comprise any carbon-to-carbon double bond or any carbon-to-carbon triple bond. A “C₁₋₆ alkyl” denotes an alkyl group having 1 to 6 carbon atoms. Preferred exemplary alkyl groups are methyl, ethyl, propyl (e.g., n-propyl or isopropyl), or butyl (e.g., n-butyl, isobutyl, sec-butyl, or tert-butyl). Unless defined otherwise, the term “alkyl” preferably refers to C₁₋₄ alkyl, more preferably to methyl or ethyl, and even more preferably to methyl. The skilled person is aware of typically used abbreviations for different alkyl groups, for example Me for methyl, Et for ethyl, Bu for butyl or tBu for tert-butyl.

As used herein, the term “aryl” refers to an aromatic hydrocarbon ring group, including monocyclic aromatic rings as well as bridged ring and/or fused ring systems containing at least one aromatic ring (e.g., ring systems composed of two or three fused rings, wherein at least one of these fused rings is aromatic; or bridged ring systems composed of two or three rings, wherein at least one of these bridged rings is aromatic). “Aryl” may, e.g., refer to phenyl, naphthyl, dialinyl (i.e., 1,2-dihydronaphthyl), tetralinyl (i.e., 1,2,3,4-tetrahydronaphthyl), indanyl, indenyl (e.g., ¹H-indenyl), anthracenyl, phenanthrenyl, 9H-fluorenyl, or azulenyl. Unless defined otherwise, an “aryl” preferably has 6 to 14 ring atoms, more preferably 6 to 10 ring atoms, even more preferably refers to phenyl or naphthyl, and most preferably refers to phenyl.

As used herein, the term “heteroaryl” refers to an aromatic ring group, including monocyclic aromatic rings as well as bridged ring and/or fused ring systems containing at least one aromatic ring (e.g., ring systems composed of two or three fused rings, wherein at least one of these fused rings is aromatic; or bridged ring systems composed of two or three rings, wherein at least one of these bridged rings is aromatic), wherein said aromatic ring group comprises one or more (such as, e.g., one, two, three, or four) ring heteroatoms independently selected from O, S and N, and the remaining ring atoms are carbon atoms, wherein one or more S ring atoms (if present) and/or one or more N ring atoms (if present) may optionally be oxidized, and further wherein one or more carbon ring atoms may optionally be oxidized (i.e., to form an oxo group). For example, each heteroatom-containing ring comprised in said aromatic ring group may contain one or two O atoms and/or one or two S atoms (which may optionally be oxidized) and/or one, two, three or four N atoms (which may optionally be oxidized), provided that the total number of heteroatoms in the corresponding heteroatom-containing ring is 1 to 4 and that there is at least one carbon ring atom (which may optionally be oxidized) in the corresponding heteroatom-containing ring. “Heteroaryl” may, e.g., refer to thienyl (i.e., thiophenyl), benzo[b]thienyl, naphtho[2,3-b]thienyl, thianthrenyl, furyl (i.e., furanyl), benzofuranyl, isobenzofuranyl, chromanyl, chromenyl (e.g., 2H-1-benzopyranyl or 4H-1-benzopyranyl), isochromenyl (e.g., ¹H-2-benzopyranyl), chromonyl, xanthenyl, phenoxathiinyl, pyrrolyl (e.g., ¹H-pyrrolyl), imidazolyl, pyrazolyl, pyridyl (i.e., pyridinyl; e.g., 2-pyridyl, 3-pyridyl, or 4-pyridyl), pyrazinyl, pyrimidinyl, pyridazinyl, indolyl (e.g., 3H-indolyl), isoindolyl, indazolyl, indolizinyl, purinyl, quinolyl, isoquinolyl, phthalazinyl, naphthyridinyl, quinoxalinyl, cinnolinyl, pteridinyl, carbazolyl, β-carbolinyl, phenanthridinyl, acridinyl, perimidinyl, phenanthrolinyl (e.g., [1,10]phenanthrolinyl, [1,7]phenanthrolinyl, or [4,7]phenanthrolinyl), phenazinyl, thiazolyl, isothiazolyl, phenothiazinyl, oxazolyl, isoxazolyl, oxadiazolyl (e.g., 1,2,4-oxadiazolyl, 1,2,5-oxadiazolyl (i.e., furazanyl), or 1,3,4-oxadiazolyl), thiadiazolyl (e.g., 1,2,4-thiadiazolyl, 1,2,5-thiadiazolyl, or 1,3,4-thiadiazolyl), phenoxazinyl, pyrazolo[1,5-a]pyrimidinyl (e.g., pyrazolo[1,5-a]pyrimidin-3-yl), 1,2-benzoisoxazol-3-yl, benzothiazolyl, benzothiadiazolyl, benzoxazolyl, benzisoxazolyl, benzimidazolyl, benzo[b]thiophenyl (i.e., benzothienyl), triazolyl (e.g., ¹H-1,2,3-triazolyl, 2H-1,2,3-triazolyl, ¹H-1,2,4-triazolyl, or 4H-1,2,4-triazolyl), benzotriazolyl, ¹H-tetrazolyl, 2H-tetrazolyl, triazinyl (e.g., 1,2,3-triazinyl, 1,2,4-triazinyl, or 1,3,5-triazinyl), furo[2,3-c]pyridinyl, dihydrofuropyridinyl (e.g., 2,3-dihydrofuro[2,3-c]pyridinyl or 1,3-dihydrofuro[3,4-c]pyridinyl), imidazopyridinyl (e.g., imidazo[1,2-a]pyridinyl or imidazo[3,2-a]pyridinyl), quinazolinyl, thienopyridinyl, tetrahydrothienopyridinyl (e.g., 4,5,6,7-tetrahydrothieno[3,2-c]pyridinyl), dibenzofuranyl, 1,3-benzodioxolyl, benzodioxanyl (e.g., 1,3-benzodioxanyl or 1,4-benzodioxanyl), or coumarinyl. Unless defined otherwise, the term “heteroaryl” preferably refers to a 5 to 14 membered (more preferably 5 to 10 membered) monocyclic ring or fused ring system comprising one or more (e.g., one, two, three or four) ring heteroatoms independently selected from O, S and N, wherein one or more S ring atoms (if present) and/or one or more N ring atoms (if present) are optionally oxidized, and wherein one or more carbon ring atoms are optionally oxidized; even more preferably, a “heteroaryl” refers to a 5 or 6 membered monocyclic ring comprising one or more (e.g., one, two or three) ring heteroatoms independently selected from O, S and N, wherein one or more S ring atoms (if present) and/or one or more N ring atoms (if present) are optionally oxidized, and wherein one or more carbon ring atoms are optionally oxidized.

Thus, the method of the present invention for preparing the compound of formula (I) may comprise the step of reacting the compound of formula (Id):

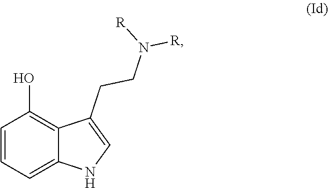

wherein each R is as defined for formula (I),
with (R′O)₂POCl, wherein each R′ is independently selected from C₁₋₆ alkyl, —CH₂-aryl or —CH₂-heteroaryl, preferably wherein each R′ is independently selected from C₁₋₆ alkyl and —CH₂-aryl, more preferably wherein each R′ is independently C₁₋₆ alkyl. Particularly suitable C₁₋₆ alkyl groups are methyl, ethyl and tert-butyl. Particularly suitable —CH₂-aryl is benzyl. Thus, in the method of the present invention, particularly preferred compounds (R′O)₂POCl are (MeO)₂POCl, (EtO)₂POCl, (tBuO)₂POCl and (benzyl-O)₂POCl, more preferably (EtO)₂POCl, (tBuO)₂POCl and (benzyl-O)₂POCl, even more preferably (EtO)₂POCl, and (tBuO)₂POCl, still more preferably (EtO)₂POCl (also referred to as DECP).

Preferably, within the scope of the present invention, the product of the reaction of the compound of formula (Id) with said (R′O)₂POCl (preferably DECP), as described hereinabove, is further reacted with TMSBr, resulting in the compound of formula (I). This reaction may also be referred to herein as deprotection reaction.

Thus, the present invention relates to a method for preparing a compound of formula (I):

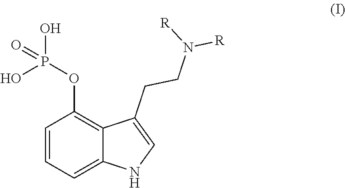

wherein each R is independently C₁₋₆ alkyl, preferably methyl or ethyl,
wherein the method comprises the step of reacting the compound of formula (Id):

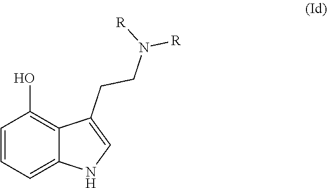

with (R′O)₂POCl, wherein each R′ is independently selected from C₁₋₆ alkyl, —CH₂-aryl and —CH₂-heteroaryl wherein the product of the reaction of the compound of formula (Id) with said (R′O)₂POCl is further reacted with TMSBr resulting in the compound of formula (I).

While in the present specification the method is described in detail for the embodiment wherein both R′ are ethyl, it is recognizable to the skilled person that the method of the present invention is generalizable to the entire provided scope of R′, that is for R′ being C₁₋₆ alkyl, —CH₂-aryl or —CH₂-heteroaryl. The embodiment of the present invention, wherein both R′ are ethyl, and for which the present invention is exemplified, is particularly preferred.

It is further apparent to the skilled person that the product of the reaction of the compound of formula (Id) with (R′O)₂POCl, wherein each R′ is independently selected from C₁₋₆ alkyl, —CH₂-aryl and —CH₂-heteroaryl, can also be obtained under different conditions, for example by reacting the compound of formula (Id) with (R′O)₂POH in the conditions leading to the presence of (R′O)₂POCl, for example in the presence of a base and tetrachloromethane, or wherein (R′O)₂POCl is generated in situ in the presence of NCS (N-chlorosuccinimide) and THF, as described in WO 2022/016289. Accordingly, when referring in the present description to a product obtainable the reaction of the compound of formula (Id) with (R′O)₂POCl, wherein each R′ is independently selected from C₁₋₆ alkyl, —CH₂-aryl and —CH₂-heteroaryl (preferably independently selected from C₁₋₆ alkyl and —CH₂-aryl, more preferably independently is C₁₋₆ alkyl), said product is not limited to a product obtained in the reaction of the compound of formula (Id) with (R′O)₂POCl, wherein each R′ is independently selected from C₁₋₆ alkyl, —CH₂-aryl and —CH₂-heteroaryl, but also includes said product obtained in any other way, for example by reacting the compound of formula (Id) with (R′O)₂POH in the conditions leading to the presence of (R′O)₂POCl, as described in WO 2022/016289.

It has been postulated by the present inventors that said product, without being bound to the theory, is a compound of formula:

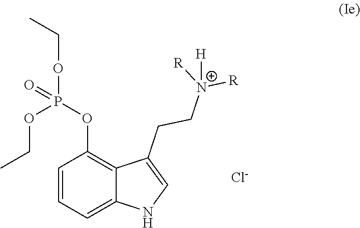

if the product is obtainable in the reaction of the compound of formula (Id) with DECP. Each R is as defined for formula (I), preferably each R is independently methyl or ethyl, It is further postulated by the present inventors that said product is a compound of formula (Ie-1):

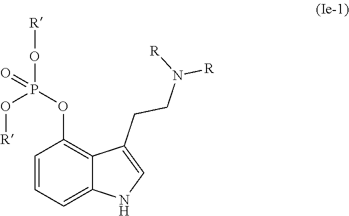

or its salt (e.g. HCl salt), if the product is obtainable in the reaction of the compound of formula (Id) with (R′O)₂POCl, as defined hereinabove. Each R is as defined for formula (I), preferably each R is independently methyl or ethyl, and each R′ is as defined for (R′O)₂POCl.

Accordingly, in view of the foregoing, the present invention relates in one embodiment to a method of obtaining the compound of formula (I):

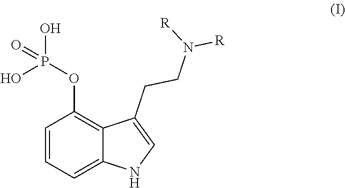

wherein each R is independently C₁₋₆ alkyl, preferably methyl or ethyl,
the method comprising the step of reacting a product obtainable in the reaction of the compound of formula (Id):

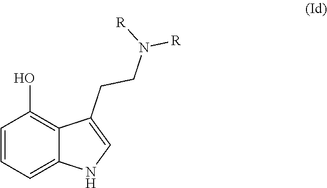

with (R′O)₂POCl, wherein each R′ is independently selected from C₁₋₆ alkyl, —CH₂-aryl and —CH₂-heteroaryl, with TMSBr resulting in the compound of formula (I).

Preferably, each R′ is independently selected from C₁₋₆ alkyl and —CH₂-aryl, More preferably, each R′ is independently C₁₋₆ alkyl. Even more preferably, both R′ are ethyl (in other words, each R′ is ethyl).

Further examples and/or embodiments of the present invention are disclosed in the following numbered items.
1. A method for preparing a compound of formula (I):

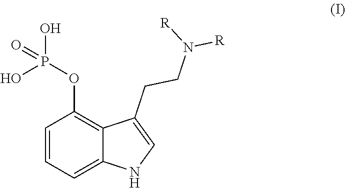

wherein each R is independently methyl or ethyl,
wherein the method comprises the step of reacting the compound of formula (Id):

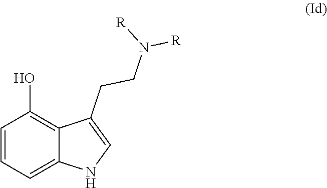

with (EtO)₂POCl (DECP).
2. The method of item 1, wherein each R is methyl.
3. The method of item 1 or 2, wherein the w/w ratio of the compound of formula (Id) and (DECP) is between 0.8 and 1.2, preferably between 1.0 and 1.2, more preferably between 1.10 and 1.15, even more preferably about 1.12, even more preferably 1.12.
4. The method of any one of items 1 to 3, wherein DECP is added dropwise over the time of between 10 and 20 minutes.
5. The method of any one of items 1 to 4, wherein the reaction of the compound of formula (Id) with DECP is performed in acetonitrile.
6. The method of any one of items 1 to 5, wherein the reaction of the compound of formula (Id) with DECP is performed at a temperature of between 50 °C and 70 °C, preferably at a temperature of between 55 °C and 65 °C, more preferably at a temperature of between 57 °C and 63 °C, even more preferably at a temperature of about 60 °C, even more preferably at a temperature of 60 °C.
7. The method of any one of items 1 to 6, wherein the reaction of the compound of formula (Id) with DECP is performed in the presence of a base, preferably wherein the base is N-ethyldiisopropylamine.
8. The method of any one of items 1 to 7, wherein the reaction of the compound of formula (Id) with DECP is performed for a time of at least 150 minutes, until at least 99% of the compound of formula (Id) has been reacted.
9. The method of any one of items 1 to 8, wherein upon the reaction of the compound of formula (Id) with DECP, the solvent is changed to toluene and aqueous NaOH solution is added thereto, followed by the phase separation.
10. The method of item 8, wherein NaOH is added at the concentration of 0.5 M to 1.0 M, preferably at the concentration of about 0.75 M, more preferably at the concentration of 0.75 M.
11. The method of any one of items 1 to 10, wherein upon completion of the reaction of the compound of formula (Id) with DECP, the solvent is changed to CPME and HCl in CPME is added to the reaction mixture, leading to precipitation of a crude product.
12. The method of item 11, wherein HCl in CPME is about 3M solution, and/or wherein said solution is added dropwise over a time of between 25 and 35 minutes, and/or wherein said solution is added at a temperature of between 15 °C and 20 °C, preferably at a temperature of between 17 °C and 23 °C, more preferably at a temperature of about 20 °C, even more preferably at a temperature of 20 °C.
13. The method of any one of items 1 to 12, wherein the reaction of the compound of formula (Id) with DECP results in a crude product of formula (Ie):

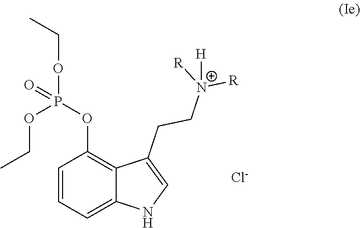

14. The method of any one of items 1 to 13, wherein the crude product precipitated from CPME upon addition of HCl comprises a compound of formula (Ie).
15. The method of any one of items 1 to 14, wherein the product of the reaction of the compound of formula (Id) with DECP is further reacted with TMSBr resulting in the compound of formula (I).
16. The method of item 15, wherein the w/w ratio of TMSBr to the compound of formula (Id) is between 1.5 and 2.5, preferably between 1.8 to 2.4, more preferably between 2.0 and 2.3, even more preferably is about 2.1, even more preferably is 2.07.
17. The method of item 15 or 16, wherein the product of the reaction of the compound of formula (Id) with DECP is reacted with TMSBr in acetonitrile.
18. The method of any one of items 15 to 17, wherein TMSBr is added at a temperature of between 30 °C to 50 °C, preferably at a temperature of between 35 °C and 45 °C, more preferably at a temperature of between 37 °C and 43 °C, even more preferably at a temperature of about 40 °C, even more preferably at a temperature of 40 °C.
19. The method of any one of items 15 to 18, wherein the product of the reaction of the compound of formula (Id) with DECP is reacted with TMSBr at a temperature of between 50 °C and 70 °C, preferably at a temperature of between 55 °C and 65 °C, more preferably at a temperature of between 57 °C and 63 °C, even more preferably at a temperature of about 60 °C, even more preferably at a temperature of 60 °C.
20. The method of any one of items 15 to 19, wherein the reaction with TMSBr is conducted for at least 150 minutes, until the product of the reaction of the compound of formula (Id) with DECP is consumed in at least 99%.
21. The method of any one of items 15 to 20, wherein upon reaction with TMSBr, the solvent is changed to methanol.
22. The method of any one of items 15 to 21, wherein the obtained product of the reaction with TMSBr is subjected to solvent change to water, wherein the pH of the solution is set to a value in the range of between pH=3.8 and pH=4.2, preferably through addition of 1 M NaOH.
23. The method of item 22, wherein the w/w ratio of 1M NaOH solution to the original amount of the compound of formula (Id) added is about 4.0, preferably is 4.0.
24. The method of item 22 or 23, wherein the final product of formula (I) precipitates from aqueous solution at pH of between 3.8 and 4.2 and/or at a temperature of less than 20 °C, preferably at a temperature of about 15 °C, more preferably at a temperature of 15 °C.
25. The method of any one of items 1 to 24, wherein the final product of formula (I) is re-slurried upon treatment with water/acetone under reflux.
26. The method of any one of items 1 to 25, further comprising the step of reducing a compound of formula (Ic):

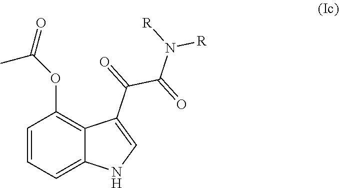

resulting in the compound of formula (Id).
27. The method of item 26 wherein in the step of reducing the compound of formula (Ic) resulting in the compound of formula (Id), LAH is used as a reducing agent.
28. The method of item 27, wherein the step of reducing the compound of formula (Ic) resulting in the compound of formula (Id) is performed in toluene, in dioxane or in CPME, preferably in toluene,
   wherein preferably said step is performed under reflux.
29. The method of item 26 wherein in the step of reducing the compound of formula (Ic) resulting in the compound of formula (Id), NaBH₄ is used as a reducing agent.
30. The method of any one of items 26 to 29, wherein the method further comprises the step of reacting a compound of formula (Ia):

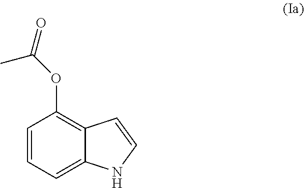

with (COCl)₂, followed by the reaction of the resulting product with R₂NH.
31. The method of item 30, wherein the reaction of the compound of formula (Ia) with (COCl)₂ is performed in CPME at a temperature of between 0 °C and 10 °C.
32. The method of item 30 or 31, wherein the reaction of the compound of formula (Ia) with (COCl)₂ results in a compound of formula (Ib):

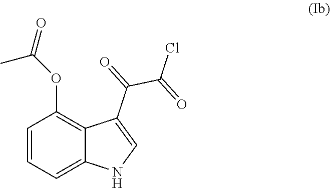 [SMILES: CC(=O)Oc1cccc2[nH]cc(C(=O)C(=O)Cl)c12]

wherein said compound of formula (Ib) is reacted with R₂NH to result in the compound of formula (Ic).

Further examples and embodiments of the present invention are disclosed in the following numbered clauses:
1. A method for preparing a compound of formula (I):

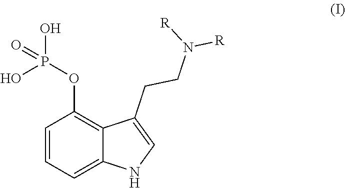

wherein each R is independently methyl or ethyl,
wherein the method comprises the step of reacting the compound of formula (Id):

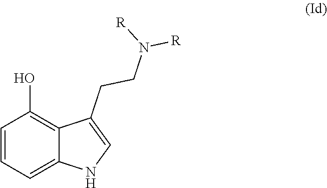

with (EtO)₂POCl (DECP).
2. The method of clause 1, wherein each R is methyl.
3. The method of clause 1 or 2, wherein the w/w ratio of the compound of formula (Id) and (DECP) is between 0.8 and 1.2, preferably between 1.0 and 1.2, more preferably between 1.10 and 1.15, even more preferably about 1.12, even more preferably 1.12,
   preferably wherein DECP is added dropwise over the time of between 10 and 20 minutes.
4. The method of any one of clauses 1 to 3, wherein the reaction of the compound of formula (Id) with DECP is performed in acetonitrile,
   preferably wherein the reaction of the compound of formula (Id) with DECP is performed at a temperature of between 50 °C and 70 °C, more preferably at a temperature of between 55 °C and 65 °C, even more preferably at a temperature of between 57 °C and 63 °C, even more preferably at a temperature of about 60 °C, even more preferably at a temperature of 60 °C,
   and/or
   wherein the reaction of the compound of formula (Id) with DECP is performed in the presence of a base, preferably wherein the base is N-ethyldiisopropylamine, and/or
   wherein the reaction of the compound of formula (Id) with DECP is performed for a time of at least 150 minutes, until at least 99% of the compound of formula (Id) has been reacted.
5. The method of any one of clauses 1 to 4, wherein upon the reaction of the compound of formula (Id) with DECP, the solvent is changed to toluene and aqueous NaOH solution is added thereto, followed by the phase separation,
   preferably wherein NaOH is added at the concentration of 0.5 M to 1.0 M, more
   preferably at the concentration of about 0.75 M, even more preferably at the concentration of 0.75 M,
   and/or
   wherein upon completion of the reaction of the compound of formula (Id) with DECP, the solvent is changed to CPME and HCl in CPME is added to the reaction mixture, leading to precipitation of a crude product,
   preferably wherein HCl in CPME is about 3M solution, and/or wherein said solution is added dropwise over a time of between 25 and 35 minutes, and/or wherein said solution is added at a temperature of between 15 °C and 20 °C, preferably at a temperature of between 17 °C and 23 °C, more preferably at a temperature of about 20 °C, even more preferably at a temperature of 20 °C.
6. The method of any one of clauses 1 to 5, wherein the reaction of the compound of formula (Id) with DECP results in a crude product of formula (Ie):

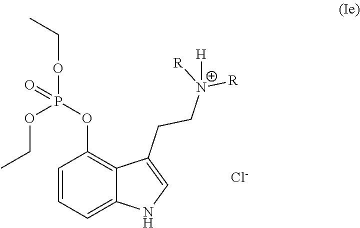

preferably wherein the crude product precipitated from CPME upon addition of HCl comprises a compound of formula (Ie).
7. The method of any one of clauses 1 to 6, wherein the product of the reaction of the compound of formula (Id) with DECP is further reacted with TMSBr resulting in the compound of formula (I),
   preferably wherein the w/w ratio of TMSBr to the compound of formula (Id) is between 1.5 and 2.5, more preferably between 1.8 to 2.4, even more preferably between 2.0 and 2.3, even more preferably is about 2.0, even more preferably is 2.07,
   preferably wherein the product of the reaction of the compound of formula (Id) with DECP is reacted with TMSBr in acetonitrile.
8. The method of clause 7, wherein TMSBr is added at a temperature of between 30 °C to 50 °C, preferably at a temperature of between 35 °C and 45 °C, more preferably at a temperature of between 37 °C and 43 °C, even more preferably at a temperature of about 40 °C, even more preferably at a temperature of 40 °C, and/or
   wherein the product of the reaction of the compound of formula (Id) with DECP is reacted with TMSBr at a temperature of between 50 °C and 70 °C, preferably at a temperature of between 55 °C and 65 °C, more preferably at a temperature of between 57 °C and 63 °C, even more preferably at a temperature of about 60 °C, even more preferably at a temperature of 60 °C,
   and/or
   wherein the reaction with TMSBr is conducted for at least 150 minutes, until the product of the reaction of the compound of formula (Id) with DECP is consumed in at least 99%.
9. The method of clause 7 or 8, wherein upon reaction with TMSBr, the solvent is changed to methanol,
   and/or
   wherein the obtained product of the reaction with TMSBr is subjected to solvent change to water, wherein the pH of the solution is set to a value in the range of between pH=3.8 and pH=4.2, preferably through addition of 1 M NaOH, preferably wherein the w/w ratio of 1M NaOH solution to the original amount of the compound of formula (Id) added is about 4.0, more preferably is 4.0, preferably wherein the final product of formula (I) precipitates from aqueous solution at pH of between 3.8 and 4.2 and/or at a temperature of less than 20 °C, preferably at a temperature of about 15 °C, more preferably at a temperature of 15 °C.
10. The method of any one of clauses 1 to 9, wherein the final product of formula (I) is re-slurried upon treatment with water/acetone under reflux.
11. The method of any one of clauses 1 to 10, further comprising the step of reducing a compound of formula (Ic):

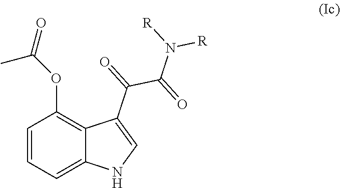

resulting in the compound of formula (Id).
12. The method of clause 11 wherein in the step of reducing the compound of formula (Ic) resulting in the compound of formula (Id), LAH is used as a reducing agent, preferably wherein the step of reducing the compound of formula (Ic) resulting in the compound of formula (Id) is performed in toluene, in dioxane or in CPME, preferably in toluene,
   wherein preferably said step is performed under reflux.
13. The method of clause 11 wherein in the step of reducing the compound of formula (Ic) resulting in the compound of formula (Id), NaBH₄ is used as a reducing agent.
14. The method of any one of clauses 11 to 13, wherein the method further comprises the step of reacting a compound of formula (Ia):

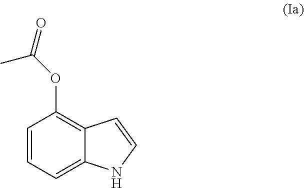

with (COCl)₂, followed by the reaction of the resulting product with R₂NH.
15. The method of clause 14, wherein the reaction of the compound of formula (Ia) with (COCl)₂ is performed in CPME at a temperature of between 0 °C and 10 °C, and/or
   wherein the reaction of the compound of formula (Ia) with (COCl)₂ results in a compound of formula (Ib):

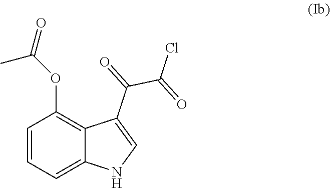 [SMILES: CC(=O)Oc1cccc2[nH]cc(C(=O)C(=O)Cl)c12]

wherein said compound of formula (Ib) is reacted with R₂NH to result in the compound of formula (Ic).

The invention will be illustrated by the following examples, which however are not to be construed as limiting.

### EXAMPLES

### Example 1: Preparation of Ethocybin According to the Invention

Herein, the scale up method for the preparation of ethocybin in accordance with the invention is described below.

Step 1: Process for the Preparation of Acetoxyindole Oxacetylchloride from 4-acetoxyindole in a Set Up as Outlined in FIG. 1 Part 1.

### Step 1a: Preparation of a Solution of Oxalylchloride in Cyclopentyl Methylether (CPME) in a 5-Litre Glass Reactor Vessel as Shown in FIG. 1.

Preparation of a solution of 4-acetoxyindole (1 volume) in 5 volumes CPME (preferably done in a 2 L glass bottle.

The 4-acetoxyindole solution was slowly added to the oxalylchloride solution under stirring. The stirring is maintained until enough 4-acetoxyindole has been converted to acetoxyindole oxacetylchloride. The reaction mixture was then concentrated under reduced pressure and repeatedly diluted with CPME and subsequently subjected to distillation (at least twice).

The resulting suspension was then diluted with n-heptane and further stirred at 0 °C in a reaction vessel as shown in FIG. 1 part 1 (Position 2). The product suspension was filtered using a 1-litre glass filter and washed twice with 2.5 volumes of n-heptane. The filter cake was then dried under a light stream of nitrogen gas.

The mass flow and the mass balance chart are shown in FIG. 1 part 3.

Step 1b: the acetoxyindole oxacetylchloride from step 1a was then dissolved in 16.9 volumes of 2-methyl tetrahydrofurane (2-MeTHF) in a 5-litre glass reaction vessel and mixed in a second 5-litre glass reaction vessel with a solution of 2.1 equivalents of diethylamine in 2-MeTHF (1 volume diethylamine and 2 volumes of 2-MeTHF) in a set up as outlined in FIG. 1 part 2.

The mixture was then further stirred until enough acetoxyindole-oxoacetylchloride has been reacted. A solution of 4.5 volumes of 1 N HCl was then added to the reaction mixture. A phase separation step was then made whereby the upper organic phase was then separated. The organic phase was then washed again with 4.5 volumes of 0.5 M HCl. The combined aqueous wash solution was then extracted twice with 4.5 volumes of 2-MeTHF and subsequently with 2.3 volumes of deionized water. Active charcoal (12.5 g) was then added to the organic phase under stirring and the mixture then filtered over 100 g of Celite (e.g. Celpure™). The so obtained organic phase was concentrated under vacuum, subjected to the cooling ramp at a temperature of −20 °C and the suspension of product was obtained (inoculation of precipitation may be necessary for obtaining the suspension). The suspension was then filtered. The so obtained filtercake was then washed twice with 1.4 volumes of 2-methyl tetrahydrofurane (MeTHF). The organic phase containing the acetoxyindole glyoxylic acid diethylamide was then further concentrated under vacuum. 163 g of acetoxyindole glyoxylic acid diethylamide was obtained.

The mass flow and the mass balance chart are shown in FIG. 1 parts 4 and 5.

The analytical data for the obtained product is shown in FIG. 5.

HPLC measurements were performed according to the following protocol:

| Gradient: | Time [min] | A [%] | B [%] |
|---|---|---|---|
| | 0.0 | 98.0 | 2.0 |
| | 2.0 | 98.0 | 2.0 |
| | 20.0 | 20.0 | 80.0 |
| | 21.0 | 20.0 | 80.0 |
| | 21.1 | 98.0 | 2.0 |
| | 25.0 | 98.0 | 2.0 |

| | |
|---|---|
| Flow: | 1 mL/min |
| Eluent A: | 0.05% TFA in water (v/v) |
| Eluent B: | 0.05% TFA in acetonitrile (v/v) |
| Column: | Poroshell 102 EC-C18, 10 cm × 4.6 mm, 4 μm |

HPLC data is shown in FIG. 5, part 1.

LC-MS was performed using mobile phase A ACN:water 1:1 using restriction capillary with pre-column at the flow of 0.5 ml (5 min). Detection was performed at the wavelength of 220 nm. LC-MS measurements (FIG. 5, parts 2 and 3) have shown the presence of two peaks: 303.2 m/z: [M+H]⁺, and 605.4 m/z: [2M+H]⁺. Further details concerning LC-MS measurements are as follows:
a. Thermofisher Vanquish ISQ Family (Single Quadrupole)
b. Heated Electrospray Ionization (HESI): Positive Mode (3000 V; 50 μA); IonTransferTube Temperature 300 °C; Vaporizer Temperature 282 °C.

¹H NMR spectrum was measured in DMSO-d6 at 400 MHz using Bruker Avance 400 spectrometer. The spectrum is shown in FIG. 5 part 4, and the observed peaks are summarized below:

| No. | Shift1 (ppm) | H's | Type | J (Hz) | Atom1 | Multiplet1 | (ppm) |
|---|---|---|---|---|---|---|---|
| 1 | 1.04 | 3 | t | 6.97 | 22, 20 | M03 | [0.99 . . . 1.09] |
| 2 | 1.16 | 3 | t | 7.10 | 22, 20 | M04 | [1.11 . . . 1.22] |
| 3 | 2.35 | 3 | s | — | 17 | M10 | [2.26 . . . 2.43] |
| 4 | 3.18 | 2 | q | 6.93 | 21, 19 | M02 | [3.12 . . . 3.23] |
| 5 | 3.41 | 2 | q | 7.10 | 21, 19 | M01 | [3.37 . . . 3.49] |
| 6 | 6.92 | 1 | dd | 7.60, 0.76 | 6 | M09 | [6.82 . . . 7.02] |
| 7 | 7.29 | 1 | t | 7.86 | 7 | M08 | [7.23 . . . 7.40] |
| 8 | 7.45 | 1 | dd | 8.11, 0.76 | 8 | M07 | [7.41 . . . 7.52] |
| 9 | 8.05 | 1 | s | — | 2 | M06 | [7.98 . . . 8.13] |
| 10 | 12.48 | 1 | br. s. | — | 1 | M05 | [12.33 . . . 12.62] |

¹³C NMR spectrum was measured in DMSO-d6 at 125 MHz using Bruker Avance 400 spectrometer. The spectrum is shown in FIG. 5 part 5, and the observed peaks are summarized below:

| No. | Shift (ppm) | Atom |
|---|---|---|
| 1 | 186.3 | 10 (C═O) |
| 2 | 169.7 | 13 (C═O) |
| 3 | 167.7 | 15 (C═O) |
| 4 | 144.5 | 5 (C—O) |
| 5 | 139.8 | 9 (C—N) |
| 6 | 139.3 | 2 (C—N) |
| 7 | 124.7 | 7 (C—H) |
| 8 | 118.7 | 4 (C) |
| 9 | 115.9 | 6 (C—H) |
| 10 | 113.2 | 3 (C) |
| 11 | 111.2 | 8 (C—H) |
| 12 | 42.0 | 19/21 (CH2) |
| 13 | 38.5 | 19/21 (CH2) |
| 14 | 21.7 | 17 (CH3) |
| 15 | 14.5 | 20/22 (CH3) |
| 16 | 13.2 | 20/22 (CH3) |

DSC measurements were performed using Mettler Toledo Thermal Analysis DSC3+ and aluminum crucible (40 μL). DSC Screening was typically performed as follows: 20-300 °C, 10 °C/min, data point 1.00 sec. DSC measurements are shown in FIG. 5, part 6.

Step 2: Process for the Preparation of 4-hydroxy-indole-3-ethyl-diethylamide (4-HO-DET) from Acetoxyindole Glyoxylic Acid Diethylamide in a Set Up as Outlined in FIG. 2 part 1.

The ketoamide from step 1b (100 g) was then combined with 15 volumes toluene in a 5-litre glass reaction vessel in a nitrogen atmosphere. An azeotropic distillation of toluene/water was then performed (at a jacket temperature (Ta) of 130±5 °C at 950 to 850 mbar under stirring (50 to 250 rpm) resulting in distillate (1) of about 3 volumes. The solution was then left to cool down to 60 °C, which has led to the precipitation of the product and formation of a suspension. 370 mL lithiumaluminiumhydride (LAH) (2.4 M) in tetrahydrofurane (THF) was then added slowly under stirring (50 to 250 rpm) at a temperature of 60 to 70 °C. The reaction mixture was then heated to a temperature of 120 °C for at least 3 hours under stirring (50 to 250 rpm) under reflux. The reaction mixture was then further stirred without refluxing. The amount of ketoamide that has reacted was determined as an in-process control step. Toluene was added again, and the reaction mixture was concentrated. The reaction mixture was cooled down to ≤55 °C. A a saturated solution of potassium sodium tartrate was added, and the reaction mixture was stirred (50 to 250 rpm) for at least 30 minutes at a temperature of 40±3 °C. Then a phase separation step was performed whereby the aqueous phase was extracted four times with toluene. The combined organic phases were washed with half-saturated potassium-sodium-tartrate solution. The aqueous phases were then back-extracted with toluene, and the obtained toluene extracts were added to the combined organic phases extracted with said potassium-sodium tartrate solution. Activated charcoal was then added under stirring to the organic phase. The mixture was then dried over sodium sulfate and clarified by filtration through Celite (e.g. Celpure™) using a 3-litre glass filter. The filtrate was then further dried over sodium sulfate and filtered again through Celite (e.g. Celpure™). The organic phase was then further concentrated. Then isopropyl acetate and n-heptane were loaded. The mixture was then left to cool down. The crystallization in the reaction mixture was then initiated and the jacket temperature (Ta) was ramped down to 0 °C during 30±5 minutes and then again during 600±30 minutes to −30 °C. The product suspension was then filtered, and the resulting filter cake was washed with isopropylactetate/n-heptane 1:1 v/v. Then the filter cake was dried under vacuum. The product 4-hydroxy-N,N-diethyltryptamine (4-HO-DET) was isolated.

The mass flow and mass balance chart are shown in FIG. 2 parts 2 and 3.

The analytical data for the obtained product is shown in FIG. 6.

HPLC measurements, performed as described hereinabove, are shown in FIG. 6 part 1.

LC-MS was performed as described hereinabove. The measurements (FIG. 6, parts 2 and 3) have shown the presence of a peak at 233.3 m/z: [M+H]⁺.

¹H NMR spectrum was measured in DMSO-d6 at 400 MHz using Bruker Avance 400 spectrometer. The spectrum is shown in FIG. 6 part 4, and the observed peaks are summarized below:

| No. | Shift1 (ppm) | H's | Type | J (Hz) | Atom1 | Multiplet1 | (ppm) |
|---|---|---|---|---|---|---|---|
| 1 | 0.95 | 6 | t | 7.10 | 14, 17 | M11 | [0.88 . . . 1.04] |
| 2 | 2.53 | 4 | m | — | 13, 16 | M10 | [2.50 . . . 2.58] |
| 3 | 2.65 | 2 | m | — | 11 | M09 | [2.61 . . . 2.72] |
| 4 | 2.84 | 2 | m | — | 10 | M08 | [2.78 . . . 2.90] |
| 5 | 6.25 | 1 | dd | 7.35, 1.01 | 6 | M06 | [6.20 . . . 6.29] |
| 6 | 6.74 | 1 | m | — | 8 | M05 | [6.69 . . . 6.76] |
| 7 | 6.80 | 1 | m | — | 7 | M04 | [6.77 . . . 6.84] |
| 8 | 6.92 | 1 | d | 2.28 | 2 | M03 | [6.87 . . . 6.99] |
| 9 | 10.59 | 1 | br. s. | — | 1 | M02 | [10.48 . . . 10.75] |
| 10 | 11.39 | 1 | s | — | 15 | M01 | [11.35 . . . 11.49] |

¹³C NMR spectrum was measured in DMSO-d6 at 125 MHz using Bruker Avance 400 spectrometer. The spectrum is shown in FIG. 6 part 5, and the observed peaks are summarized below:

| No. | Shift (ppm) | Atom |
|---|---|---|
| 1 | 152.2 | 5 (C—O) |
| 2 | 139.2 | 9 (C—N) |
| 3 | 122.4 | 7 (C—H) |
| 4 | 121.7 | 2 (C—H) |
| 5 | 117.7 | 4 (C) |
| 6 | 113.4 | 3 (C) |
| 7 | 104.4 | 6 (C—H) |
| 8 | 103.1 | 8 (C—H) |
| 9 | 55.8 | 11 (CH2) |
| 10 | 47.3 | 13/16 (CH2) |
| 11 | 25.1 | 10 (CH2) |
| 12 | 11.6 | 14/17 (CH3) |

DSC measurements, performed as described hereinabove, are shown in FIG. 6, part 6.

Step 3: Process for the Preparation of Ethocybin Crude Product from 4-HO-diethyltryptamine (4-HO-DET) via DET-Diethylphosphate×HCl as Intermediate in a Set Up as Outlined in FIG. 3 Part 1.

### Step 3a: Phosphorylation Step

The 4-HO-DET (50 g) obtained from step 2 was loaded into a 1-litre glass reaction vessel as shown in FIG. 4 with 197 g acetonitrile and the mixture was then heated to a batch temperature (TI) of 60 °C. 55.6 g N-ethyldiisopropylamine was then added. After adding diethyl chlorophosphate the mixture was further stirred at a TI of 60 °C. The amount of remaining 4-HO-DET was determined as an in-process control step. The solvent was then changed to toluene. The reaction mixture was then cooled down from room temperature to a TI of 0 °C. Then 0.75 M caustic sodium was added. A phase separation step was then performed whereby the aqueous phase was twice extracted with toluene. The combined organic phases were first washed with 0.1 N NaOH and then twice with deionized water. Active charcoal was then added to the organic phase under stirring, then dried over sodium sulfate and the mixture clarified by filtration over Celite (e.g. Celpure™ P1000) using toluene in the washing step. The solvent was then changed to cyclopentyl methyl ether (CPME). 3 M HCl in CPME was then added and the suspension was then again filtered over Celite (e.g. Celpure™ P1000) to isolate the intermediate HCl salt. The filter cake was then washed twice with CPME. The amount of the resulting TET-diethylphosphate×HCl was then determined as an in-process control step. The filter cake was then dried further under reduced pressure.

### Step 3b: Deprotection

The DET-diethyl phosphate×HCl was loaded into the reaction vessel in the presence of acetonitrile. The reaction mixture was then heated to a batch temperature (TI) of 40 °C. Bromotrimethylsilane was then added and the reaction mixture heated to a TI of 60 °C under stirring. The amount of remaining DET-diethyl phosphate×HCl was determined as an in-process control step. The solvent was then changed to methanol. The reaction mixture was then stirred at a temperature of 50 °C. The solvent was then distilled away. The residue was again mixed with methanol and the mixture further stirred at a temperature of 50 °C. A solvent change to water was then performed. The pH of the mixture was then adjusted to a pH of between 3.8 and 4.2 with 1 N NaOH. Isopropanol was loaded and the reaction mixture was then concentrated under reduced pressure. A temperature ramp step was then performed to a jacket temperature (Ta) set point of 15 °C. The resulting suspension was then filtered. The filter cake was then successively washed first with deionized water, then with a deionized water/methanol mixture and finally with methanol alone. The resulting product was then dried under reduced pressure. The resulting product was collected and the yield of the crude ethocybin product was then determined.

The mass of flow and the mass balance chart are shown in FIG. 3 parts 2 to 4.

The analytical data for the obtained product is shown in FIG. 7.

HPLC measurements, performed as described hereinabove, are shown in FIG. 7 part 1.

LC-MS was performed as described hereinabove. The measurements (FIG. 7, parts 2 and 3) have shown the presence of a peak at 313.3 m/z: [M+H]⁺, 527.4 m/z: [2M-HPO₄]⁺, and 625.4 m/z: [2M+H]⁺.

DSC measurements, performed as described hereinabove, are shown in FIG. 7 part 4).

Step 4: Process for the Preparation of Ethocybin from the Crude Ethocybin Product from Step 3b in a Set Up as Outlined in FIG. 4.

The ethocybin raw product (80 g of step 3b) was loaded into an oxygen free (inert gas) 1 liter glass reaction vessel as specified in FIG. 5. 40 g deionized water and 62.5 g acetone were added successively, and the mixture was then stirred (at 50 to 250 rpm) at a jacket temperature set point (Ta) of 65±3 °C for at least 16 hours. The suspension was then cooled down to Ta=30 °C. The product suspension was then filtered through a 1-litre glass nutsch, The wet filter cake was washed twice first with 80 g water/acetone mixture then with 62.5 g acetone alone, whereby the wash fluid should stay with the filter cake for at least 5 minutes. The washed filter cake was then separated by filtration and then first dried by blowing nitrogen gas over it and then further dried in a vacuum chamber under reduced pressure at a temperature of 40 °C The yield of ethocybin was then determined to be 72.0 g.

The analytical data for the obtained product is shown in FIG. 8.

HPLC measurements, performed as described hereinabove, are shown in FIG. 8 part 1.

LC-MS was performed as described hereinabove. The measurements (FIG. 8, parts 2 and 3) have shown the presence of a peak at at 313.3 m/z: [M+H]⁺, 527.4 m/z: [2M−HPO₄]⁺, and 625.4 m/z: [2M+H]⁺.

¹H NMR spectrum was measured in DMSO-d6+trifluoroacetic acid at 400 MHz using Bruker Avance 400 spectrometer. The spectrum is shown in FIG. 8 part 4, and the observed peaks are summarized below:

| No. | Shift1 (ppm) | H's | Type | J (Hz) | Atom1 | Multiplet1 | (ppm) |
|---|---|---|---|---|---|---|---|
| 1 | 0.97 | 6 | t | 7.22 | 19, 14 | M07 | [0.85 . . . 1.09] |
| 2 | 2.93 | 6 | m | — | 18, 13, 11 | M06 | [2.80 . . . 3.01] |
| 3 | 3.06 | 2 | m | — | 10 | M05 | [3.01 . . . 3.14] |
| 4 | 6.73 | 1 | d | 7.86 | 6 | M04 | [6.68 . . . 6.77] |
| 5 | 6.86 | 1 | t | 7.98 | 7 | M03 | [6.82 . . . 6.90] |
| 6 | 6.93 | 1 | s | — | 2 | M02 | [6.91 . . . 6.97] |
| 7 | 7.01 | 1 | d | 8.11 | 8 | M01 | [6.97 . . . 7.06] |

¹³C NMR spectrum was measured in DMSO-d6+trifluoroacetic acid at 125 MHz using Bruker Avance 400 spectrometer. The spectrum is shown in FIG. 8 part 5, and the observed peaks are summarized below:

DSC measurements, performed as described hereinabove, are shown in FIG. 8, part 6.

### Example 2: Preparation of Psilocybin According to the Invention

The complete reaction scheme for obtaining psilocybin according to the methods of the present invention is shown in FIG. 9.

Step 1b—Acetoxyindole Glyoxylic Acid Dimethylamide

### Process Description:

Acetoxyindole oxacetylchloride (20 g) (obtained in Step 1 of Example 1, described hereinabove) was dissolved in 2-MeTHF (261.8 g, 13.1 vol.)
Dimethylamine 2 m in THF (63.8 g, 2.0 eq) loaded in reactor and cooled to Ti: −10 to −5 °C
solution of the acid chloride added to the amine solution maintaining the inner temp. at Ti: −10 to 5 °C
container used for the acid chloride solution rinsed with 2-MeTHF (27.2 g, 1.6 vol.) into the reaction vessel
sample for IPC taken (stirring reaction mass at Ti: 0 °C until result of the IPC available)
reaction mass concentrated under reduced pressure at Ta: 40 °C to approx. 5 vol.
2-MeTHF (193 g, 10.8 vol) was added to the reaction mass
reaction mass again concentrated to approx. 5 vol.
2-MeTHF (177 g, 10 vol) was added to the reaction mass, thereby THF was (at least to an extent) removed and replaced with 2-MeTHF (according to the present inventors, presence of THF had an adverse effect on the workup).
0.5 m HCl (4 vol.) were slowly added to reaction mixture maintaining the inner temperature at Ti: 0 to 15 °C
if the reaction mass is a suspension: reaction mass filtered and filter cake washed with 2-MeTHF/water 1:1 v/v (2.5 vol.); filter cake dried at Ta: 40 °C under reduced pressure to obtain a first crop of the product (approx. 30% yield)
combined mother- and washing liquor loaded in the reactor and phases separated
aqueous phase extracted with ethyl acetate (5 vol.) (ethyl acetate shown in this case improved phase separation when compared to 2-Me-THF, which was used in Example 1 concerning ethocybin)
combined organic phases washed with NaHCO₃ (5 vol.) twice
organic phase loaded in the reactor and concentrated under reduced pressure at Ta: 60 °C to approx. 2.5 vol.
cooling ramp: Ta: 0 °C within 60 min and post stirring for 60 in at Ta: 0 °C
suspension filtered off and filter cake washed with ethyl acetate (1 vol.)
filter cake dried under reduced pressure at Ta: 40 °C
second crop approx. 22%

Analytical data for the so obtained product is shown in FIG. 10.

### Step 2: Psilocin (4-Hydroxy-dimethyltrypthamine)

### Process Description:

The same process as for the synthesis of Ethocin (4-HO-DET) in Example 1 was used, except for:
CPME as reaction solvent
no charcoal treatment in the work up
yield: 70%

Analytical data for the obtained product is shown in FIG. 11.

### Step 3: Psilocybin

### Process Description:

the same process as for the synthesis of Ethocybin (see Example 1 hereinabove) was used
yield: 25%

Analytical data for the so obtained product is shown in FIG. 12.

1. A method for preparing a compound of formula (I): 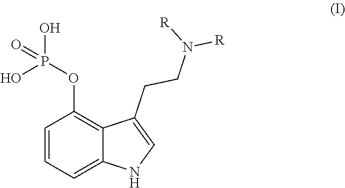
wherein each R is independently C₁₋₆ alkyl, preferably methyl or ethyl,
wherein the method comprises the step of reacting the compound of formula (Id) 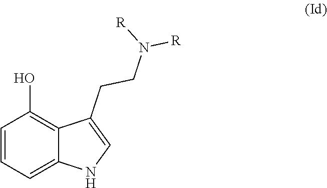
with (R′O)₂POCl, wherein each R′ is independently selected from C₁₋₆ alkyl, —CH₂-aryl and —CH₂-heteroaryl,
wherein the product of the reaction of the compound of formula (Id) with said (R′O)₂POCl is further reacted with TMSBr, resulting in the compound of formula (I).

2. The method of claim 1, wherein (R′O)₂POCl is (MeO)₂POCl, (EtO)₂POCl, (tBuO)₂POCl or (benzyl-O)₂POCl, more preferably (EtO)₂POCl or (tBuO)₂POCl, even more preferably (EtO)₂POCl.

3. The method of claim 1 or 2 for preparing a compound of formula (I): 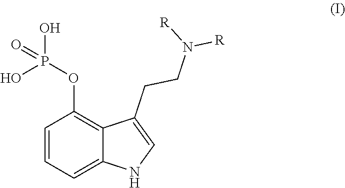
wherein each R is independently C₁₋₆ alkyl, preferably methyl or ethyl,
wherein the method comprises the step of reacting the compound of formula (Id): 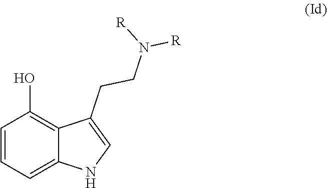
with (EtO)₂POCl (DECP),
wherein the product of the reaction of the compound of formula (Id) with DECP is further reacted with TMSBr, resulting in the compound of formula (I).

4. The method of any one of claims 1 to 3, wherein each R is independently methyl or ethyl.

5. The method of any one of claims 1 to 3, wherein each R is methyl.

6. The method of any one of claims 1 to 3, wherein each R is ethyl.

7. The method of claim 3 or any one of claims 4 to 6 insofar dependent on claim 3, wherein the w/w ratio of the compound of formula (Id) and (DECP) is between 0.8 and 1.2, preferably between 1.0 and 1.2, more preferably between 1.10 and 1.15, even more preferably about 1.12, even more preferably 1.12,
preferably wherein DECP is added dropwise over the time of between 10 and 20 minutes.

8. The method claim 3 or 7, or any one of claims 4 to 6 insofar dependent on claim 3, wherein the reaction of the compound of formula (Id) with DECP is performed in acetonitrile.

9. The method of claim 8, wherein the reaction of the compound of formula (Id) with DECP is performed at a temperature of between 50 °C and 70 °C, preferably at a temperature of between 55 °C and 65 °C, more preferably at a temperature of between 57 °C and 63 °C, even more preferably at a temperature of about 60 °C, yet even more preferably at a temperature of 60 °C.

10. The method of claim 8 or 9, wherein the reaction of the compound of formula (Id) with DECP is performed in the presence of a base, preferably wherein the base is N-ethyldiisopropylamine.

11. The method of any one of claims 8 to 10, wherein the reaction of the compound of formula (Id) with DECP is performed for a time of at least 150 minutes, until at least 99% of the compound of formula (Id) has been reacted.

12. The method of any one of claims 3 or 7 to 11, or any one of claims 4 to 6 insofar dependent on claim 3, wherein upon the reaction of the compound of formula (Id) with DECP, the solvent is changed to toluene and aqueous NaOH solution is added thereto, followed by the phase separation,
preferably wherein NaOH is added at the concentration of 0.5 M to 1.0 M, more preferably at the concentration of about 0.75 M, even more preferably at the concentration of 0.75 M.

13. The method of any one of claims 3 or 7 to 12, or any one of claims 4 to 6 insofar dependent on claim 3, wherein upon completion of the reaction of the compound of formula (Id) with DECP, the solvent is changed to CPME and HCl in CPME is added to the reaction mixture, leading to precipitation of a crude product,
preferably wherein HCl in CPME is about 3M solution, and/or wherein said solution is added dropwise over a time of between 25 and 35 minutes, and/or wherein said solution is added at a temperature of between 15 °C and 20 °C, preferably at a temperature of between 17 °C and 23 °C, more preferably at a temperature of about 20 °C, even more preferably at a temperature of 20 °C.

14. The method of any one of claims 3 or 7 to 13, or any one of claims 4 to 6 insofar dependent on claim 3, wherein the reaction of the compound of formula (Id) with DECP results in a crude product of formula (Ie): 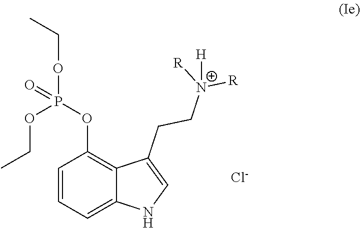
preferably wherein the crude product precipitated from CPME upon addition of HCl comprises a compound of formula (Ie).

15. The method of any one of claims 1 to 14, wherein the w/w ratio of TMSBr to the compound of formula (Id) is between 1.5 and 2.5, more preferably between 1.8 to 2.4, even more preferably between 2.0 and 2.3, even more preferably is about 2.0, even more preferably is 2.07.

16. The method of any one of claims 1 to 15, wherein the product of the reaction of the compound of formula (Id) with (R′O)₂POCl, preferably with DECP, is reacted with TMSBr in acetonitrile.

17. The method of any one of claims 1 to 16, wherein TMSBr is added at a temperature of between 30 °C to 50 °C, preferably at a temperature of between 35 °C and 45 °C, more preferably at a temperature of between 37 °C and 43 °C, even more preferably at a temperature of about 40 °C, even more preferably at a temperature of 40 °C.

18. The method of any one of claims 1 to 17, wherein the product of the reaction of the compound of formula (Id) with (R′O)₂POCl, preferably with DECP, is reacted with TMSBr at a temperature of between 50 °C and 70 °C, preferably at a temperature of between 55 °C and 65 °C, more preferably at a temperature of between 57 °C and 63 °C, even more preferably at a temperature of about 60 °C, even more preferably at a temperature of 60 °C.

19. The method of any one of claims 1 to 18, wherein the reaction with TMSBr is conducted for at least 150 minutes, until the product of the reaction of the compound of formula (Id) with (R′O)₂POCl, preferably with DECP, is consumed in at least 99%.

20. The method of any one of claims 1 to 19, wherein upon reaction with TMSBr, the solvent is changed to methanol.

21. The method of any one of claims 1 to 20, wherein the obtained product of the reaction with TMSBr is subjected to solvent change to water, wherein the pH of the solution is set to a value in the range of between pH=3.8 and pH=4.2, preferably through addition of 1 M NaOH, preferably wherein the w/w ratio of 1M NaOH solution to the original amount of the compound of formula (Id) added is about 4.0, more preferably is 4.0, preferably wherein the final product of formula (I) precipitates from aqueous solution at pH of between 3.8 and 4.2 and/or at a temperature of less than 20 °C, preferably at a temperature of about 15 °C, more preferably at a temperature of 15 °C.

22. The method of any one of claims 1 to 21, wherein the final product of formula (I) is re-slurried upon treatment with water/acetone under reflux.

23. The method of any one of claims 1 to 22, further comprising the step of reducing a compound of formula (Ic): 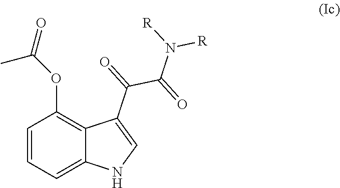
resulting in the compound of formula (Id).

24. The method of claim 23 wherein in the step of reducing the compound of formula (Ic) resulting in the compound of formula (Id), LAH is used as a reducing agent, preferably wherein the step of reducing the compound of formula (Ic) resulting in the compound of formula (Id) is performed in toluene, in dioxane or in CPME, preferably in toluene,
wherein preferably said step is performed under reflux.

25. The method of claim 23 wherein in the step of reducing the compound of formula (Ic) resulting in the compound of formula (Id), NaBH₄ is used as a reducing agent.

26. The method of any one of claims 23 to 25, wherein the method further comprises the step of reacting a compound of formula (Ia): 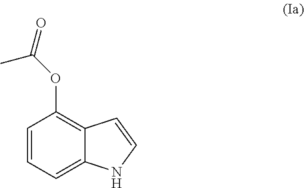
with (COCl)₂, followed by the reaction of the resulting product with R₂NH.

27. The method of claim 26, wherein the reaction of the compound of formula (Ia) with (COCl)₂ is performed in CPME at a temperature of between 0 °C and 10 °C, and/or
wherein the reaction of the compound of formula (Ia) with (COCl)₂ results in a compound of formula (Ib): 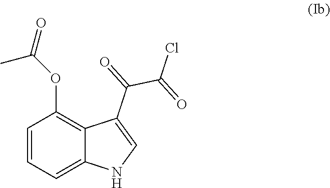 [SMILES: CC(=O)Oc1cccc2[nH]cc(C(=O)C(=O)Cl)c12]
wherein said compound of formula (Ib) is reacted with R₂NH to result in the compound of formula (Ic).

28. A method of obtaining the compound of formula (I): 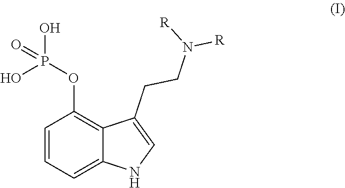
wherein each R is independently C₁₋₆ alkyl, preferably methyl or ethyl,
the method comprising the step of reacting a product obtainable in the reaction of the compound of formula (Id): 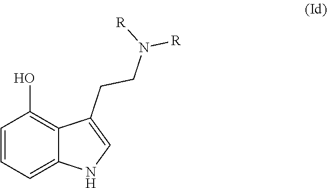
with (R′O)₂POCl, wherein each R′ is independently selected from C₁₋₆ alkyl, —CH₂-aryl and —CH₂-heteroaryl, with TMSBr, resulting in the compound of formula (I).

29. The method of claim 28, wherein each R′ is independently C₁₋₆ alkyl or —CH₂-aryl, preferably wherein each R′ is independently C₁₋₆ alkyl, more preferably wherein both R′ are ethyl.

30. The method of claim 28 of 29, wherein the product obtainable in the reaction of the compound of formula (Id) with (R′O)₂POCl is a compound of formula: 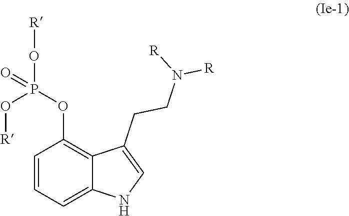
or its salt, preferably HCl salt, wherein R and R′ are as defined in claim 28 or 29.
